(19) United States
(12) Patent Application Publication
(10) Pub. No.: US 2006/0281180 A1
(43) Pub. Date: Dec. 14, 2006

(54) Vectors

(76) Inventors: Philippa Radcliffe, Oxford (GB); Fraser Wilkes, Oxford (GB); Susan Kingsman, Oxford (GB); Kyriacos Mitrophanous, Oxford (GB)

Correspondence Address: FROMMER LAWRENCE & HAUG, 745 FIFTH AVENUE- 10TH FL., NEW YORK, NY, 10151, US

(21) Appl. No.: 11/410,669

(22) Filed: Apr. 25, 2006

Related U.S. Application Data
(63) Continuation-in-part of application No. PCT/GB04/04553, filed on Oct. 28, 2004.

(30) Foreign Application Priority Data
Oct. 30, 2003 (GB) 0325379.6

(51) Int. Cl. C12N 15/867

(52) U.S. Cl. 435/456

(57) ABSTRACT

Provided is a lentiviral vector capable of delivering a nucleotide of interest (NOI) to a desired target site and wherein the NOI encodes the Factor VIII and the Factor VIII is expressed following delivery of the NOI to the desired target site.

### CROSS-REFERENCE TO RELATED APPLICATIONS

This application is a continuation-in-part of International Application No. PCT/GB2004/004553, filed Oct. 28, 2004, published as WO 2005/052171 on Jun. 9, 2005, and claiming priority to GB Application Serial No. 0325379.6, filed Oct. 30, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a vector. In particular, the present invention relates to a novel system for packaging and expressing genetic material in a retroviral particle.

### BACKGROUND OF THE INVENTION

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and packaging machinery required to make more virus, which can leave the cell by a process sometimes called “budding”.

Each virus comprises genes called gag, pol and env which code for virion proteins and enzymes. In the provirus, the retroviral genome is flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

The control of proviral transcription remains largely with the noncoding sequences of the viral LTR. The site of transcription initiation is at the boundary between U3 and R in the left hand side LTR and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins. Some retroviruses have any one or more of the following genes such as tat, rev, tax and rex that code for proteins that are involved in the regulation of gene expression.

Transcription of proviral DNA recreates the full length viral RNA genomic and subgenomic-sized RNA molecules that are generated by RNA processing. Typically, all RNA products serve as templates for the production of viral proteins. The expression of the RNA products is achieved by a combination of RNA transcript splicing and ribosomal frameshifting during translation.

RNA splicing is the process by which intervening or “intronic” RNA sequences are removed and the remaining “exonic” sequences are ligated to provide continuous reading frames for translation. The primary transcript of retroviral DNA is modified in several ways and closely resembles a cellular mRNA. However, unlike most cellular mRNAs, in which all introns are efficiently spliced, newly synthesised retroviral RNA must be diverted into two populations. One population remains unspliced to serve as the genomic RNA and the other population is spliced to provide subgenomic RNA.

The complex retroviruses, which direct the synthesis of both singly and multiply spliced RNA, regulate the transport and splicing of the different genomic and subgenomic-sized RNA species through the interaction of sequences on the RNA with the protein product of one of the accessory genes, such as rev in HIV-1.

Retroviruses are often used as a delivery system (otherwise expressed as a delivery vehicle or delivery vector) for inter alia the transfer of a NOI, or a plurality of NOIs, to one or more sites of interest. The transfer can occur in vitro, ex vivo, in vivo, or combinations thereof. When used in this fashion, the retroviruses are typically called retroviral vectors or recombinant retroviral vectors. Retroviral vectors have even been exploited to study various aspects of the retrovirus life cycle, including receptor usage, reverse transcription and RNA packaging (reviewed by Miller, 1992 Curr Top Microbiol Immunol 158:1-24).

In a typical recombinant retroviral vector for use in gene therapy, at least part of one or more of the gag, pol and env protein coding regions may be removed from the virus. This makes the retroviral vector replication-defective. The removed portions may even be replaced by a NOI in order to generate a virus capable of integrating its genome into a host genome but wherein the modified viral genome is unable to propagate itself due to a lack of structural proteins. When integrated in the host genome, expression of the NOI occurs—resulting in, for example, a therapeutic and/or a diagnostic effect. Thus, the transfer of a NOI into a site of interest is typically achieved by: integrating the NOI into the recombinant viral vector; packaging the modified viral vector into a virion coat; and allowing transduction of a site of interest—such as a targeted cell or a targeted cell population.

It is possible to propagate and isolate quantities of retroviral vectors (e.g. to prepare suitable titres of the retroviral vector) for subsequent transduction of, for example, a site of interest by using a combination of a packaging or helper cell line and a recombinant vector.

In some instances, propagation and isolation may entail isolation of the retroviral gag, pol and env genes and their separate introduction into a host cell to produce a “packaging cell line”. The packaging cell line produces the proteins required for packaging retroviral DNA but it cannot bring about encapsidation due to the lack of a psi region. However, when a recombinant vector carrying a NOI and a psi region is introduced into the packaging cell line, the helper proteins can package the psi-positive recombinant vector to produce the recombinant virus stock. This can be used to transduce cells to introduce the NOI into the genome of the cells. The recombinant virus whose genome lacks all genes required to make viral proteins can transduce only once and cannot propagate. These viral vectors which are only capable of a single round of transduction of target cells are known as replication defective vectors. Hence, the NOI is introduced into the host/target cell genome without the generation of potentially harmful retrovirus. A summary of the available packaging lines is presented in “Retroviruses” (1997 Cold Spring Harbour Laboratory Press Eds: J M Coffin, S M Hughes, H E Varmus pp 449).

There has been considerable interest in the development of lentiviral vector systems. This interest arises firstly from the notion of using HIV-based vectors to target anti-HIV therapeutic genes to HIV susceptible cells and secondly from the prediction that, because lentiviruses are able to infect non-dividing cells (Lewis & Emerman 1993 J. Virol. 68, 510), vector systems based on these viruses would be able to transduce non-dividing cells (e.g. Vile & Russel 1995 Brit. Med. Bull. 51, 12). Vector systems based on HIV have been produced (Buchschacher & Panganiban 1992 J. Virol. 66, 2731) and they have been used to transduce CD4+ cells and, as anticipated, non-dividing cells (Naldini et al, 1996 Science 272, 263). In addition lentiviral vectors enable very stable long-term expression of the gene of interest. This has been shown to be at least one year for transduced rat neuronal cells in vivo (Biennemann et al, 2003 Mol. Ther. 5, 588). The MLV based vectors were only able to express the gene of interest for six weeks.

Sometimes, in the production of lentiviral vectors it is desirable not to express the therapeutic gene in the producer cell, as this may cause a reduction in the viral titre through a number of mechanisms. In order to prevent this it is possible to adopt a split intron configured vector as described in our WO99/15683 and WO00/56910. However, expression levels from LTR promoters are generally lower than from internal promoters.

Haemophilia A affects one in every 5,000 males and is caused by a deficiency of the Factor VIII protein in the plasma. Based on the level of Factor VIII activity in the blood, haemophilia A is categorized into mild, moderate, and severe forms. Fifty percent of haemophilia A patients have the severe form of the disease that is characterized by spontaneous and prolonged bleeding episodes.

Factor VIII is a cofactor in the coagulation pathway. Circulating in the blood, Factor VIII is non-covalently complexed with its carrier protein von Willebrand factor. This interaction stabilizes Factor VIII and prevents the association of Factor VIII with membrane surfaces. The conversion of Factor VIII into its active state, Factor VIIIa, occurs via the proteolysis of Factor VIII by thrombin or Factor Xa. Human Factor VIII is synthesized as a single chain polypeptide, with a predicted molecular weight of 265 kDa. The Factor VIII gene codes for 2351 amino acids, and the protein is processed within the cell to yield a heterodimer primarily comprised of a heavy chain of 200 kDa containing the A1, A2, and B domains and an 80 kDa light chain containing the A3, C1, and C2 domains (Kaufman et al., J. Biol. Chem., 263:6352-6362 [1988]). Both the single chain polypeptide and the heterodimer circulate in the plasma as inactive precursors (Ganz et al., Eur. J. Biochem., 170:521-528 [1988]). Activation of Factor VIII in plasma is initiated by thrombin cleavage between the A2 and B domains, which releases the B domain and results in a heavy chain consisting of the A1 and A2 domains. The proteolysed Factor VIIIa dissociates from von Willebrand Factor. A membrane bound complex containing Factor VIIIa and Factor IXa is formed that subsequently activates Factor X in the coagulation cascade. Haemophilia may result from point mutations, deletions, or mutations resulting in a stop codon (See, Antonarakis et al., Mol. Biol. Med., 4:81 [1987]).

Currently, haemophilia A is treated by the frequent infusion of purified Factor VIII into the blood. While this method of treating haemophilia A does reduce the frequency and severity of bleeding, this therapy is limited by the availability and the cost of purified Factor VIII, the short half life of Factor VIII in vivo, and the necessity of removing contaminating AIDS and hepatitis viruses. While recombinant Factor VIII is now available, this form of Factor VIII maintenance therapy is both expensive and chronic.

Gene therapy is an attractive alternative to the protein infusion treatments for haemophilia A. Two gene therapy approaches may be used. In vivo gene therapy introduces nucleotides encoding the Factor VIII protein into the patient's cells. Ex vivo gene therapy techniques introduce the nucleotides encoding the Factor VIII protein into in vitro cultured cells. The transformed cultured cells are subsequently reimplanted into the patient.

Studies of Factor VIII biogenesis and secretion have been limited by the lack of human cell lines that express significant amounts of Factor VIII. Analysis of secretion has been limited to autologous gene expression. In general, these studies show Factor VIII has low expression levels. See, for example, Lenting et al. (1998) Blood 92:3983-3996, Connelly et al. (1996) Human Gene Therapy 7:183-195, Kaufman et al. (1989) Mol. Cell. Biol. 9: 1233, Dorner et al. (1987) J. Cell Biol. 105:2665 and the references cited therein.

Human and canine studies have shown that Factor VIII levels rise to normal following liver transplantation, during which there can be no extrahepatic synthesis of Factor VIII. This indicates that the liver synthesizes a clinically significant amount of Factor VIII protein. It is well known in the art that hepatocytes express Factor VIII, however, whether other types of liver cells synthesize Factor VIII remains controversial. See, for reviews, Bloom et al. (1979) Clin. Haematol. 8:53-77 and Lenting (1998) Blood 92:3983-3996, both of which are herein incorporated by reference.

Many different gene therapy approaches to treat haemophilia A are currently being studied. Ex vivo gene therapy techniques have found that Factor VIII protein expression is low in transduced in vitro cultured cells and undetectable in vivo (Lynch et al. (1993) Hum. Gene Therapy 4:259; Chuah et al. (1995) Hum. Gene Ther. 6:1363; Hoeben et al. (1990) J. Biol. Chem. 265:7318; Hoeben et al. (1993) Hum. Gene Ther. 4:179; Israel et al. (1990) Blood 75:1074 and van der Eb (1996) J. Clin. Biochem. Nutr. 21: 78-80; all of which are herein incorporated by reference). This suggests that there is a need to develop constructs which allow higher levels of Factor VIII expression.

U.S. Pat. Nos. 6,221,349 and 6,200,560 both disclose gene therapy constructs containing Factor VIII in adeno-associated virus vectors.

Although it is known in the literature that inclusion of the Factor VIII gene within retroviral vectors has often resulted in low vector titre this has generally been ascribed to transcriptional silencers within the gene and/or the lack of an intron upstream of the gene. The interference of functional viral particle production as a result of expression of the Factor VIII protein within producer cells has not been reported. That this has not previously been discovered in light of the large number of studies in this field is surprising.

### SUMMARY OF THE INVENTION

The present invention seeks to provide a novel retroviral vector capable of providing efficient expression of a nucleotide of interest (NOI)—or even a plurality of NOIs—at one or more target sites.

The present invention also seeks to provide a novel system for efficiently preparing titres of virion vector which incorporate safety features for in vivo use and which is capable of providing efficient expression of an NOI—or even a plurality of NOIs—at one or more target sites.

In one embodiment the vector of this invention can be used to treat haemophilia. In particular it provides a way in which lentiviral based Factor VIII expression vectors can be produced at titres high enough for effective gene therapy. In another aspect it allows Factor VIII to be expressed under tissue specific promoters (for example a liver specific promoter).

According to one aspect of the present invention there is provided a lentiviral vector capable of delivering a nucleotide of interest (NOI) to a desired target site and wherein the NOI encodes for Factor VIII and the Factor VIII is only expressed at the desired target site.

According to another aspect of the present invention there is provided a retroviral vector comprising a nucleotide sequence encoding for and capable of expressing Factor VIII wherein the nucleotide sequence is operably linked to a tissue specific promoter.

Expression of Factor VIII following transfection of the cDNA into mammalian cells is reported to be two to three orders of magnitude lower than generally obtained with other genes. Kaufman et al (1989 Mol. Cell Biol. 9: 1233-42) reported three different reasons for this:
1. Inefficient expression of the Factor VIII mRNA.
2. Inefficient transport of the primary translation product from the Endoplasmic Reticulum to the Golgi apparatus.
3. The requirement for high levels of von Willebrands'Factor (vWF) to promote stable accumulation of the protein.

Various factors have been proposed which may limit accumulation of Factor VIII mRNA in transfected cells including transcriptional attenuation (Hoeben et al 1995 Blood 85: 2447-54; Koeberl et al 1995 Human Gene Ther. 6: 469-79; Fallaux et al 1996 Mol. Cell Biol. 16: 4264-72). However, Kaufman et al (1989 ibid) proposed that the major rate-limiting step was at a post-transcriptional level. The inclusion of an intron upstream of Factor VIII has been found to significantly improve expression (Chuah et al 1995 Human Gene Ther. 6: 1363-77; Dwarki et al 1995 Proc Natl Acad. Sci. USA 92: 1023-7; Chuah et al 1998 Human Gene Ther. 9: 353-65; VandenDriessche et al 1999 Proc Natl Acad. Sci. USA 96: 10379-84).

According to another aspect of the present invention there is provided a polynucleotide sequence encoding Factor VIII and which is codon optimised for efficient expression in a mammalian cell.

The rationale for codon-optimising the Factor VIII gene was to improve translational efficiency. Significant enhancement of Factor VIII mRNA accumulation, through elimination of inhibitory elements, was thought unlikely as this strategy has previously been tried and was unsuccessful: conserved mutagenesis of the putative 1.2 kb inhibitory region failed to yield a significant increase in Factor VIII expression (Chuah et al 1995 ibid). Indeed, the very existence of transcriptional inhibitory elements has been called into question (Kaufman, 1999 Human Gene Ther. 10: 2091-107). Codon-optimisation has been very successful in improving the expression of genes from viruses such as HIV-1 GagPol (Kotsopoulou et al 2000 J. Virol. 74: 4839-52) and Cre recombinase (Koresawa 2000 Transplant Proc. 32: 2516-7), bacteria, for example the tetracycline repressor (Wells 1999 Transgenic Res. 8: 371-81), and the green fluorescent protein from the jellyfish Aequorea Victoria (Haas et al 1996 Curr Biol. 6: 315-24). As these organisms are highly diverged from mammals re-engineering these genes to conform to the codon bias of highly expressed human proteins might be expected to result in a substantial improvement in expression. Mammalian genes do not show such profound codon bias as do genes from, for example Escherichia.

Nevertheless, as a poorly expressed gene, we decided to re-engineer the codons of the Factor VIII gene. The translational efficiency of the Factor VIII mRNA was previously found to be comparable to that of two other mRNAs tested: vWF and dihydrofolate reductase (Kaufman et al, 1989 ibid), therefore, it was anticipated that enhancement of gene expression would likely be modest. Despite this it was considered that this would be a worthwhile approach as any improvement in expression of the gene would be useful in the development of a haemophilia A gene therapeutic.

Surprisingly, we have found that codon optimisation has improved the expression of Factor VIII approximately 20-fold. The magnitude of the improvement is surprising in light of the following:
1. Factor VIII is a human gene, hence any benefit would be predicted to be modest compared to re-engineering a viral or bacterial genes, or a gene from a different species.
2. A similar strategy (conserved mutagenesis of nearly a quarter of the cDNA) previously failed to improve expression.
3. Translation of the mRNA has been studied and was not found to be inefficient.

In a highly preferred embodiment, codon optimisation was based on the codon usage of highly expressed human genes (Haas et al 1996, Curr. Biol. 6, 315). See table for Factor VIII genes shown in FIG. 15. Preferred embodiments of the codon optimised Factor VIII gene are shown in FIG. 19 and FIG. 21 (bases 20 to 7072).

According to another aspect of the present invention there is provided a retroviral vector capable of delivering a first nucleotide of interest (NOI) and derivable from a retroviral pro-vector, wherein the retroviral pro-vector comprises a first NOI operably linked to an internal promoter and a second NOI between the first NOI and the internal promoter such that the second NOI is capable of being spliced out, and wherein the promoter, first NOI and second NOI are in reverse complement orientation and optionally wherein the second NOI is out of frame with respect to the first NOI.

In preferred embodiments the viral vector genomes employed with the codon-optimised Factor VIII and/or the Factor VIII operably linked to a tissue specific promoter have at least one of more of the following features:
1. WPRE present
2. major splice donor mutated
3. partial Tat ORF disrupted
4. to minimise any possible read-through from upstream ORFs, Factor VIII ORFs may be cloned out of frame.

This invention concerns a vector construct which allows recombinant vectors to be produced in packaging cells without the therapeutic gene being expressed. This is achieved by inserting an intron, containing an ORF (open reading frame) or at least part thereof, which is preferably out of frame, optionally with its own promoter, between the promoter and the therapeutic gene. The ORF may code for any gene including, but not limited to, reporter genes such as lac Z and GFP or antibiotic resistance genes. The ORF is also in the reverse complement orientation and, as it is the first ORF encountered downstream of the internal promoter, by the translation machinery it is translated before the therapeutic gene. Translation stops at the end of the ORF at the stop signal. In order to further minimise the likelihood of the therapeutic gene being expressed, a polyadenylation signal (also within the intron) may be added after the first ORF. This will aid translation termination as well as reducing transcription of the reverse complement strand beyond this point.

In order for the first NOI to be expressed in the target cells, it is necessary for the ORF within the intron to be removed in the vector genome transcript. This is ensured by the presence of a splice donor and splice acceptor site flanking this region in the correct orientation for splicing of the genome transcript prior to packaging. In the presence of rev, the intron remains in place. In the absence of rev the intron is spliced out, thereby also removing the ORF. In target cells transduced by the latter the therapeutic gene will be expressed as normal. In other words, the strategy exploits the ability to produce vectors in the absence of rev. The protein encoded by the ORF, and not the therapeutic, will be expressed in the producer cell. However, the ORF will be spliced out of the genome transcript prior to packaging. As the first ORF has been spliced out of the genome transcript, the therapeutic gene will be expressed in the transduced cells following integration.

In accordance with the present invention, each NS can be any suitable nucleotide sequence. For example, each sequence can be independently DNA or RNA—which may be synthetically prepared or may be prepared by use of recombinant DNA techniques or may be isolated from natural sources or may be combinations thereof. The sequence may be a sense sequence or an antisense sequence. There may be a plurality of sequences, which may be directly or indirectly joined to each other, or combinations thereof.

The second NOI may include any one or more of the following selectable markers which have been used successfully in retroviral vectors: the bacterial neomycin and hygromycin phosphotransferase genes which confer resistance to G418 and hygromycin respectively (Palmer et al 1987 Proc Natl Acad Sci 84: 1055-1059; Yang et al 1987 Mol Cell Biol 7: 3923-3928); a mutant mouse dihydrofolate reductase gene (dhfr) which confers resistance to methotrexate (Miller et al 1985 Mol Cell Biol 5: 431-437); the bacterial gpt gene which allows cells to grow in medium containing mycophenolic acid, xanthine and aminopterin (Mann et al 1983 Cell 33: 153-159); the bacterial hisD gene which allows cells to grow in medium without histidine but containing histidinol (Danos and Mulligan 1988 Proc Natl Acad Sci 85: 6460-6464); the multidrug resistance gene (mdr) which confers resistance to a variety of drugs (Guild et al 1988 Proc Natl Acad Sci 85: 1595-1599; Pastan et al 1988 Proc Natl Acad Sci 85: 4486-4490) and the bacterial genes which confer resistance to puromycin or phleomycin (Morgenstern and Land 1990 Nucleic Acid Res 18: 3587-3596).

All of these markers are dominant selectable markers and allow chemical selection of most cells expressing these genes. GFP/β-galactosidase can also be considered a dominant marker; cells expressing GFP/β-galactosidase can be selected by using the fluorescence-activated cell sorter. In fact, any cell surface protein can provide a selectable marker for cells not already making the protein. Cells expressing the protein can be selected by using the fluorescent antibody to the protein and a cell sorter. Other selectable markers that have been included in vectors include the hprt and HSV thymidine kinase which allows cells to grow in medium containing hypoxanthine, amethopterin and thymidine.

The second NOI could contain non-coding sequences that render the first NOI non-translational in the packaging cells (for example a polyadenylation signal) but when they are removed by splicing, following transduction the first NOI is subsequently revealed for functional expression.

The second NOI may also encode a viral essential element such as env encoding the Env protein which can reduce the complexity of production systems.

Suitable first NOI coding sequences include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppressor protein and growth factors, membrane proteins, vasoactive proteins and peptides, anti-viral proteins and ribozymes, and derivatives thereof (such as with an associated reporter group).

The first NOI coding sequence may encode a fusion protein or a segment of a coding sequence.

### BRIEF DESCRIPTION OF THE DRAWINGS

FIG. 1 shows a schematic of a vector according to one aspect of the present invention. SD=splice donor, SA=splice acceptor, pA=polyadenylation signal, BGH=bovine growth hormone, syn=synthetic, =packaging signal.

FIG. 2 shows a schematic of an integrated vector according to one aspect of the present invention.

FIG. 3 shows amino acid sequence flanking the Factor VIII B-domain. In more detail, A2 sequence (from 737 to 740; SEQ ID NO:19), A3 sequence (from 1690 to 1696; SEQ ID NO:20). The sites cleaved by thrombin during proteolytic activation are shown (boxed). The SQ version of Factor VIII was created by fusion of Ser743 to Gln1638 whereas the LA version was created by deletion of residues 760 through 1639 (fusing Thr759 to Pro1640). Arg740 and Glu1649 are assumed to be important for processing of Factor VIII. The SQ version therefore has a link of 14 amino acids between the C-terminus (Arg740) of the 90 kDa chain and the N-terminus of the 80 kDa light chain.

FIG. 4 shows a schematic of human α1-antitrypsin promoter (305 bp) (Kramer et al (2003) Mol Ther. 7:375-85). In more detail, Specific (C/EBP, CCAAT enhancer binding protein α or β; HNF, hepatocyte nuclear factor) and nonspecific (AP-1) activating transcription factors are indicated. Binding regions of putative repressor factors present in nonhepatic cells are depicted (De Simone and Cortese 1989). Coordinates with respect to the cap site are indicated. Regulatory elements are shown: DE, distal element; TSE, tissue-specific element, TATA box.

FIG. 5 shows predicted titres of viral vector preparations as measured by PERT (performance enhanced reverse transcription) assay. Vector genomes express LacZ or Factor VIII from the CMV promoter.

FIG. 6 shows RNA genome levels of vectors with CMV and tissue-specific promoters. In more detail, predicted titres of vectors expressing GFP, LacZ and Factor VIII from either the hAAT (dark) or ICAM-2 (light) promoters. Vectors containing the internal CMV promoter were also prepared alongside as controls (NCG=pONY8.95NCG, NCZ=pONY8.95NCZ, NCF=pONY8.7NCF). Vectors were pseudotyped with Ross River Virus (RRV) or Ebola envelopes.

FIG. 7 shows promoter activity in 293T cells. In more detail, 293T cells transfected with genomes expressing GFP from different internal promoters (indicated) and viewed by phase contrast or UV microscopy.

FIG. 8 shows HepG2 and 293A cells transduced with vectors as indicated.

FIG. 9 shows HUVEC cells transduced with indicated vectors.

FIG. 10 shows the results of an integration assay: hAAT and CMV promoters. In more detail, 293A cells were transduced with the indicated vectors (RRV-pseudotyped). Following passage and DNA extraction, EIAV Ψ levels were measured by real-time PCR.

FIG. 11 shows the results of an integration assay: ICAM2 and CMV promoters. In more detail, 293A cells were transduced with the indicated vectors (Ebola-pseudotyped). Following passage and DNA extraction, EIAV Ψ levels were measured by real-time PCR.

FIG. 12 shows pONY8.95NCZ (VSV-G) titres when co-transfected with a second genome. In more detail, equal quantities of the pONY8.95NCZ plasmid and the plasmid indicated were used in transfections. Resulting LacZ titres are shown.

FIG. 13 shows D17 titres of HIV, MLV and EIAV: Factor VIII genome mixing. In more detail, HIV (pH7G), MLV (pHIT111) and EIAV (pONY8.7NCZ) vectors were prepared by transfection using optimised ratios of plasmid components. To the transfection mix was added 2 μg of the plasmid indicated. D17 titres (colony forming units) are shown.

FIG. 14 shows D17 titres of pONY8.4NCZ (SINMIN) vectors with mutation of Tat Exon 1 and/or major splice donor 1.

FIG. 15 shows a codon usage table for Factor VIII genes.

FIG. 16 shows the results of a COATEST.

FIG. 17 shows a comparison of wild type and codon optimised Factor VIII genes by protein quantity and activity assays.

FIG. 18 shows a Western blot of supernatants from HepG2s transduced with EIAV vectors encoding Factor VIII (lane 1: untransduced; lane 2: CO×1; lane 3: CO×1; lane 4: WT×10; lane 5: untransduced; lane 6: pONY8.95 NAF; lane 7: pONY8.95NAF; lane 8: marker; lane 9: marker; lane 10: rFVIII).

FIG. 19 shows a codon-optimised Factor VIII nucleotide sequence (SEQ ID NO:21) in accordance with the present invention.

FIG. 20 shows a diagram of the full-length, codon-optimised Factor VIII gene in the pONY8.95 backbone designated pONY8.95NAFβ.

FIG. 21 shows the complete sequence of pONY8.95NAFβ(SEQ ID NO:22).

FIG. 22 shows the translation (SEQ ID NO:24) of the full length, codon-optimised sequence (SEQ ID NO:23).

FIG. 23 shows a comparison of titres for pONY8.95-hAAT vectors containing codon optimised full length Factor VIII (NAFb), wild type Factor VIII (NAFa), B-domain deleted Factor VIII (NASqwt) and codon optimised B-domain deleted Factor VIII (NAF).

FIG. 24 shows the affect of expression of Factor VIII in 293T producer cells on VSV-G envelope concentration.

FIG. 25 shows the affect of Factor VIII expression on production of viral vector production when pseudotyped with different envelope proteins.

### DETAILED DESCRIPTION

Various preferred features and embodiment of the present invention will now be described by way of non-limiting example.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Factor VIII Genes

The present invention preferably involves the use of a therapeutic NOI which gives rise to human Factor VIII or a homologue or functional derivative thereof. A sequence for functional human factor VIII is given in U.S. Pat. No. 5,618,788.

In one embodiment we constructed the full length codon optimised Factor VIII gene.

There are a number of B-domain deleted Factor VIII gene derivatives; i.e. derivatives in which the B-domain molecule to which no essential function has been ascribed is deleted, and which may be used in the present invention.

In one embodiment, we based the synthetic gene on the ‘LA’ version which has been well-characterised biochemically (Pittman et al 1993). A precursor of this construct, pDGR-2 (Toole et al 1986) was ordered from the LGC (ATCC # 53100) to enable comparison of wild type and codon-optimised genes. Both codon-optimised and wild-type versions of the two genes were constructed.

In another embodiment we constructed a shorter ‘SQ’ version from the synthetic gene by overlapping PCR.

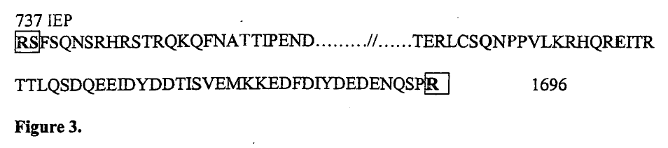

Amino acid sequence flanking the Factor VIII B-domain is shown is FIG. 3.

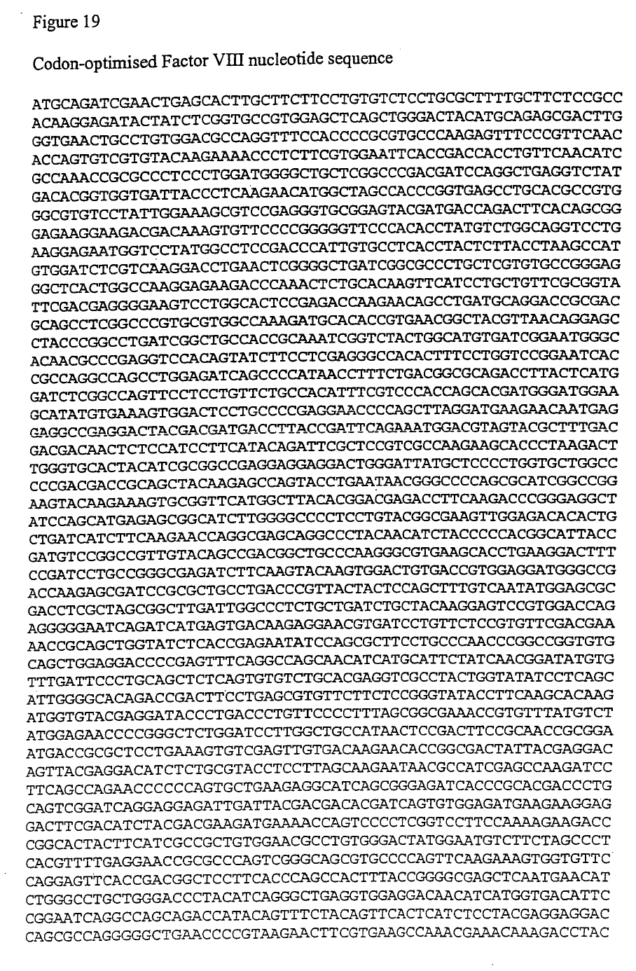

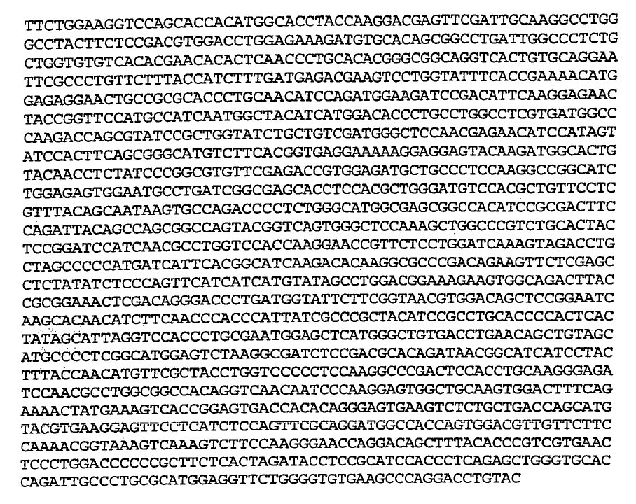

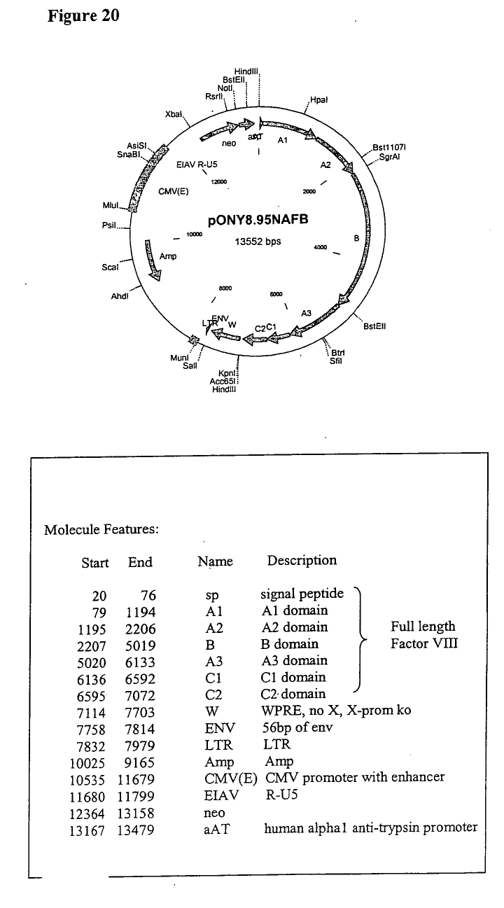

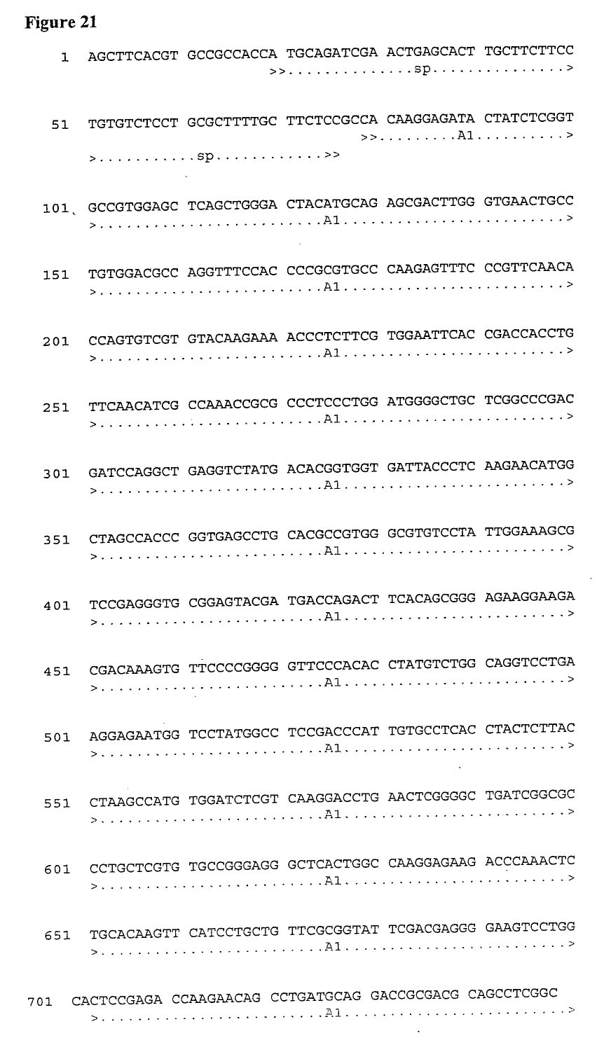

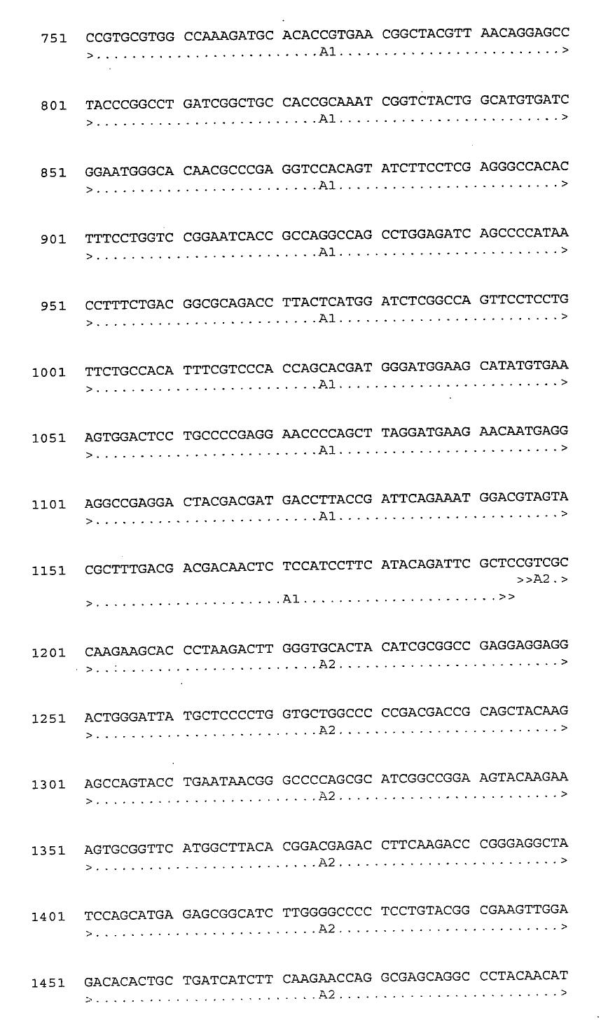

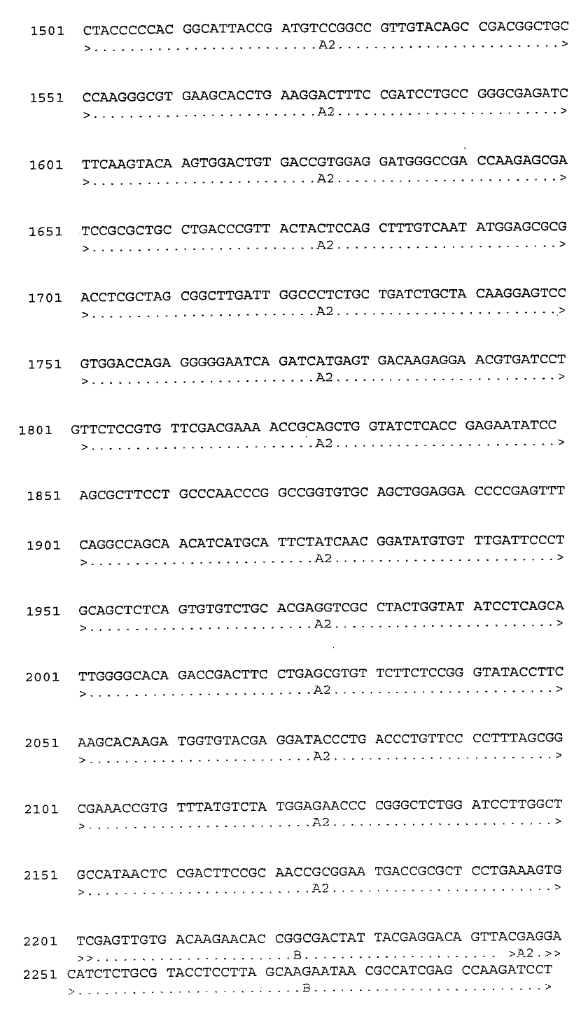

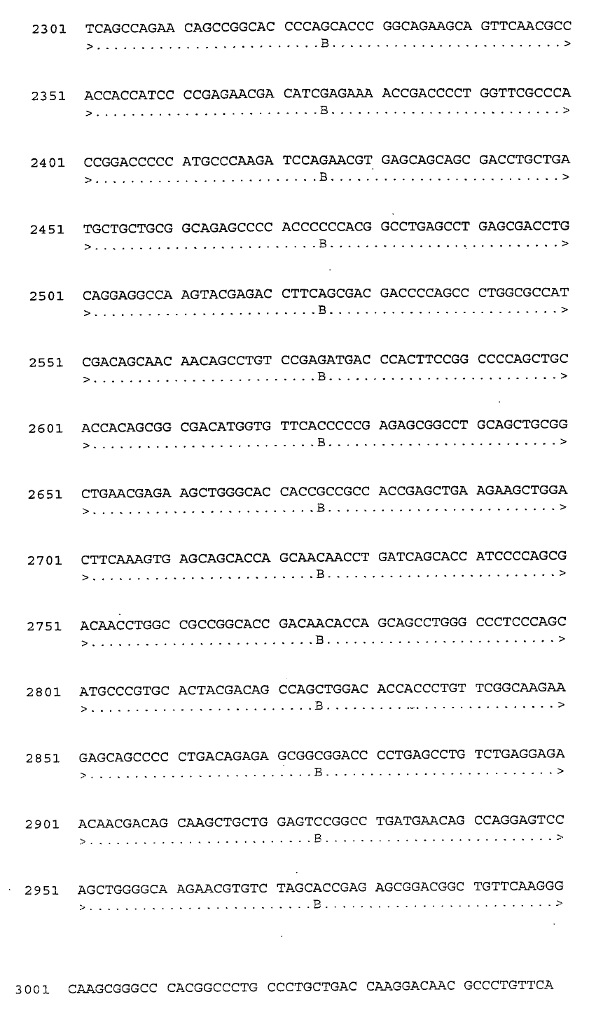

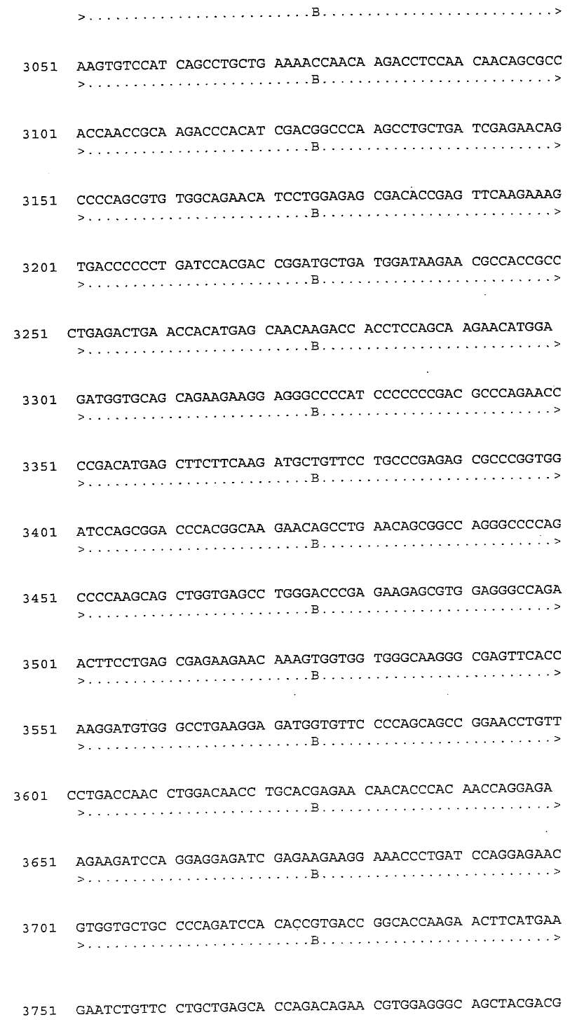

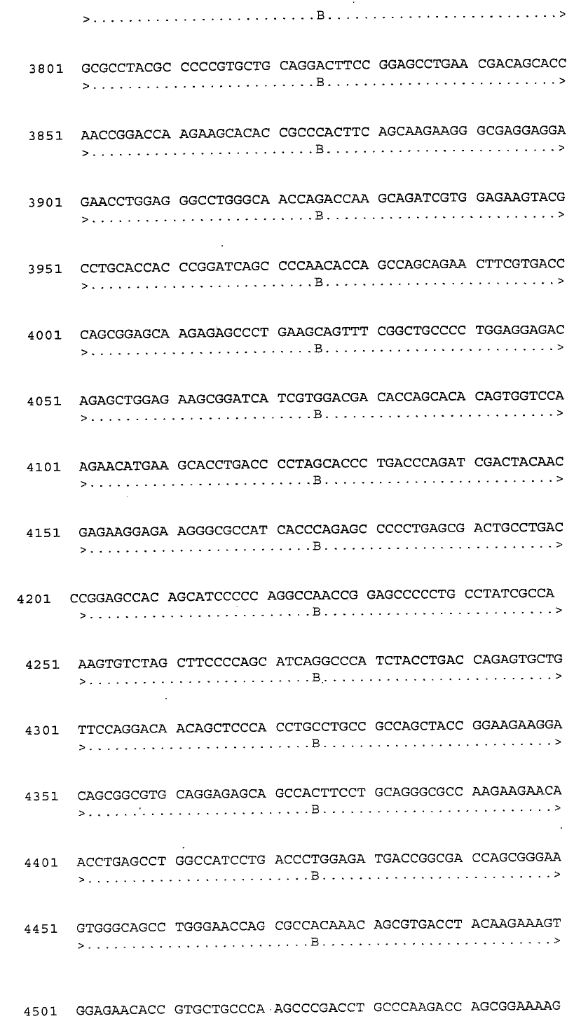

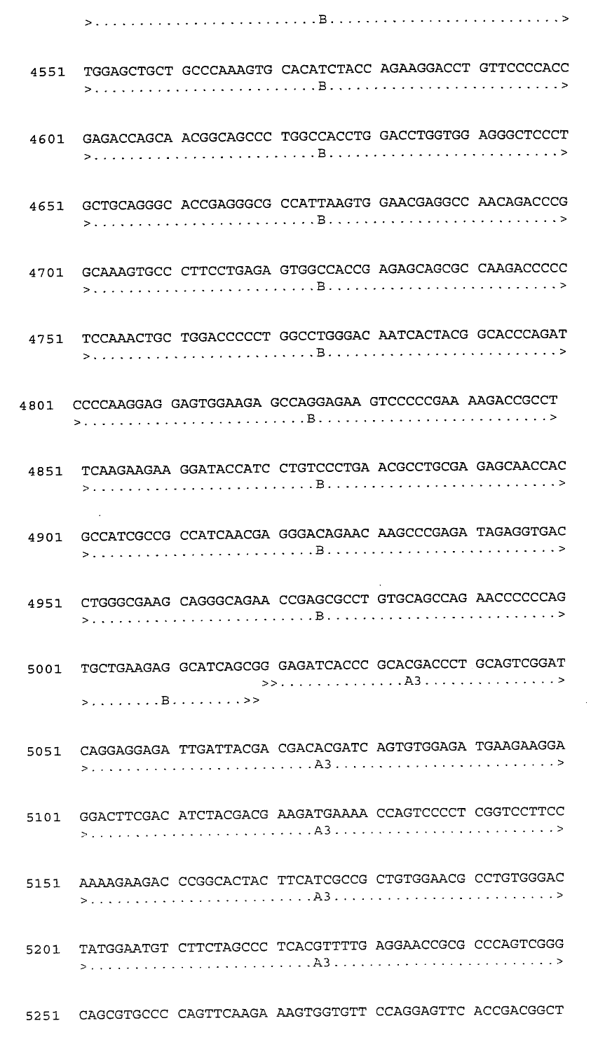

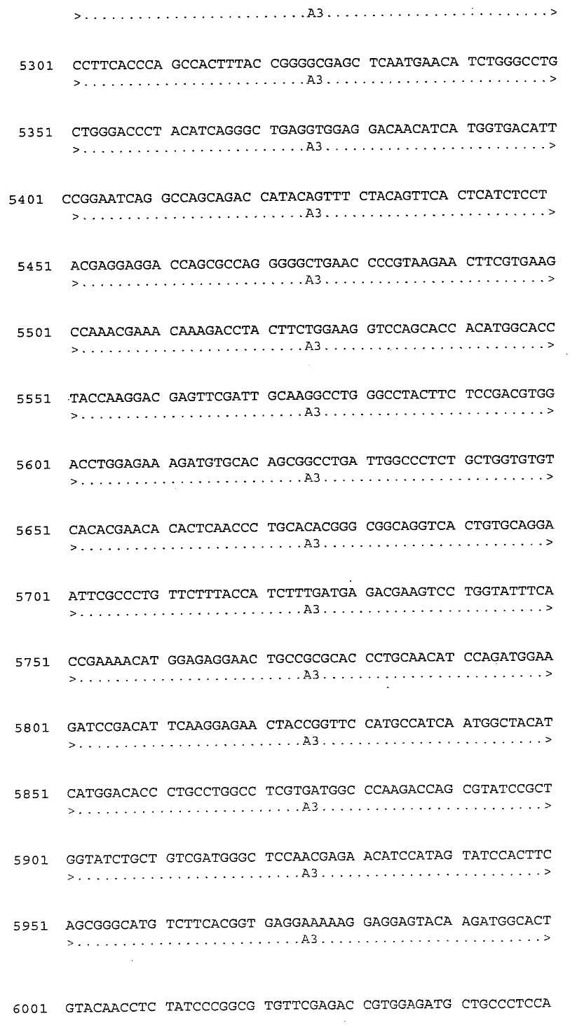

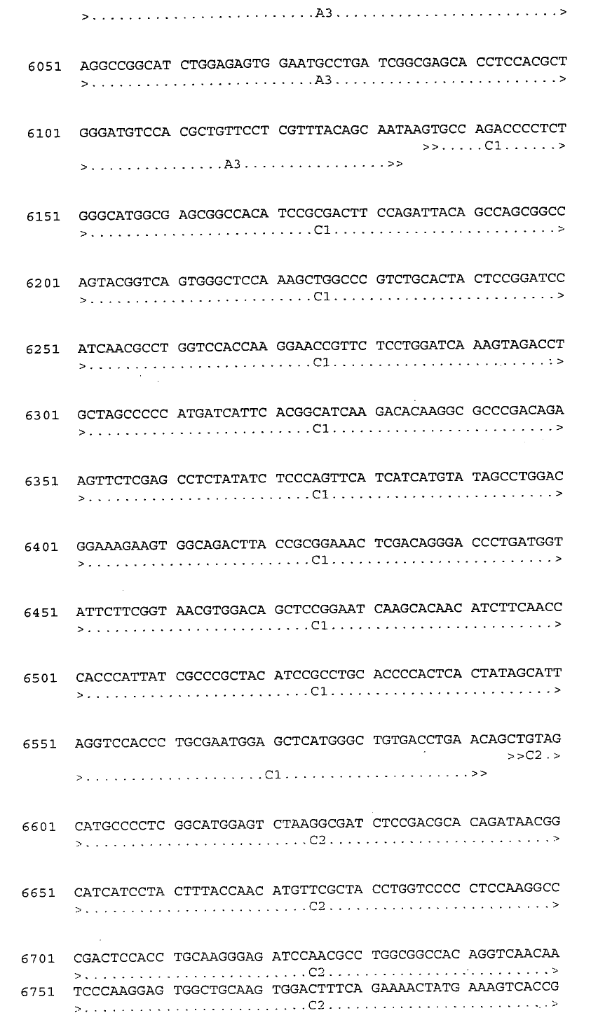

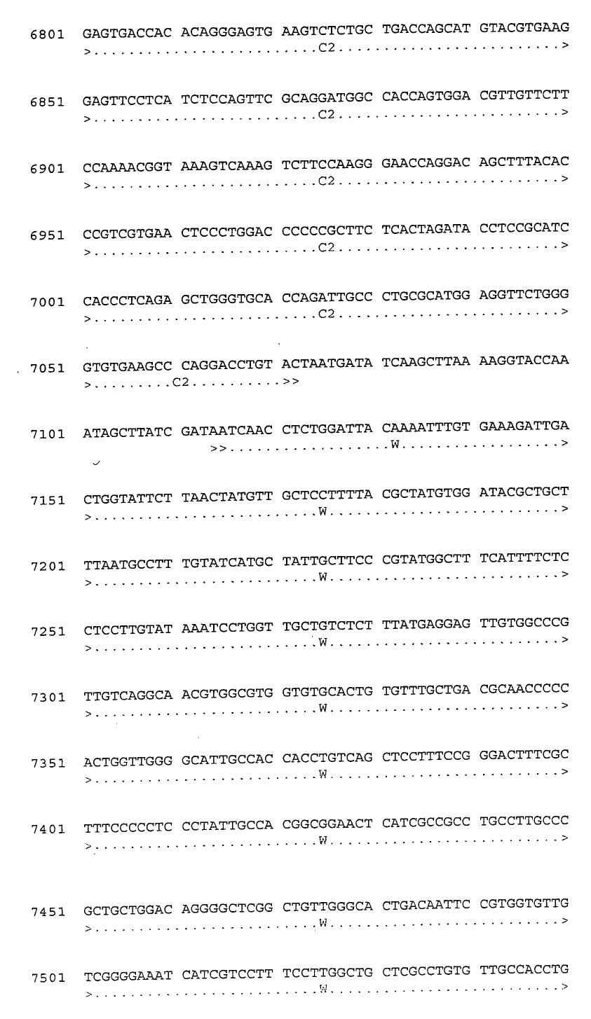

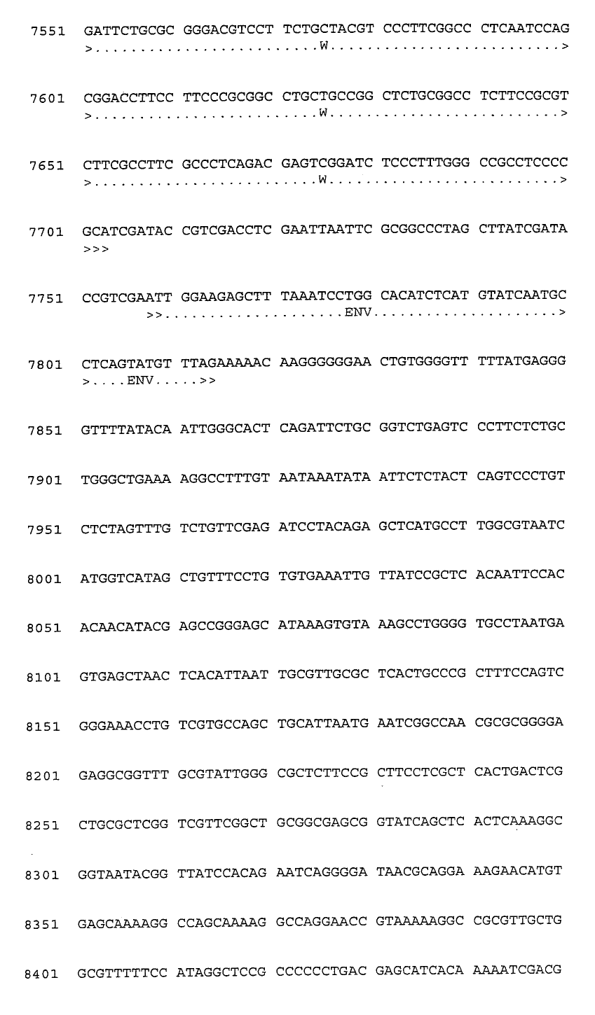

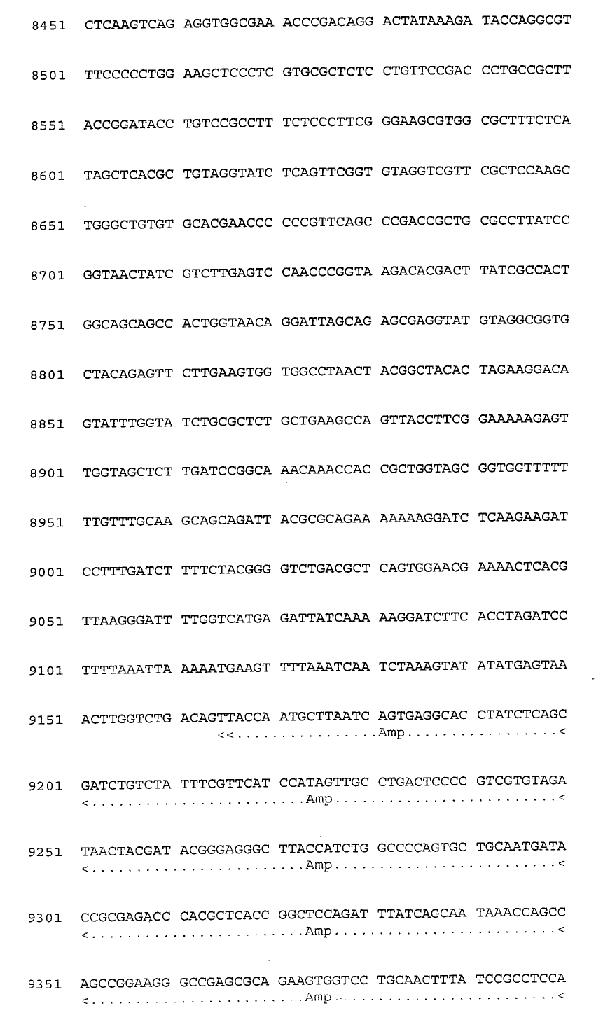

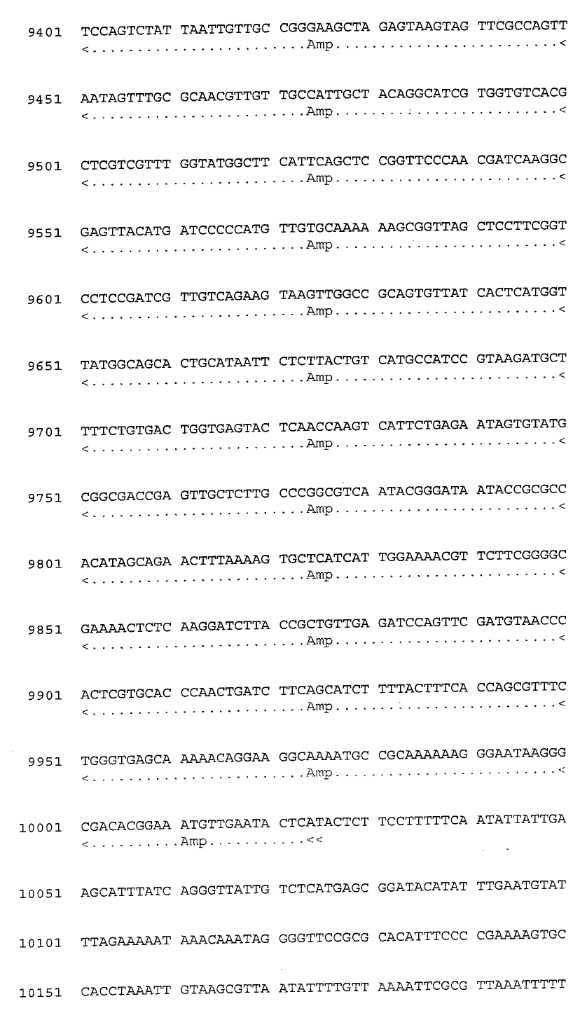

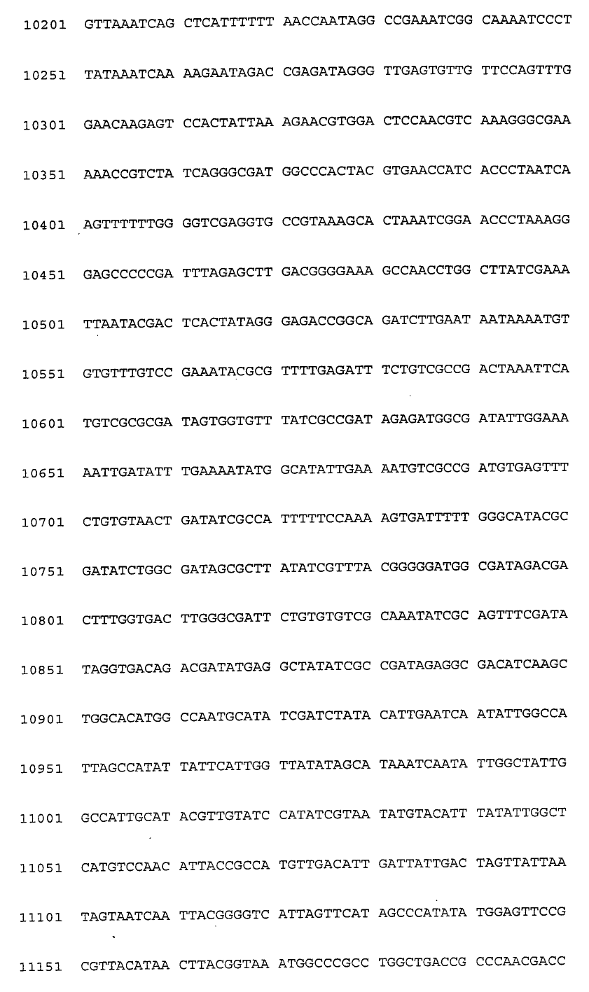

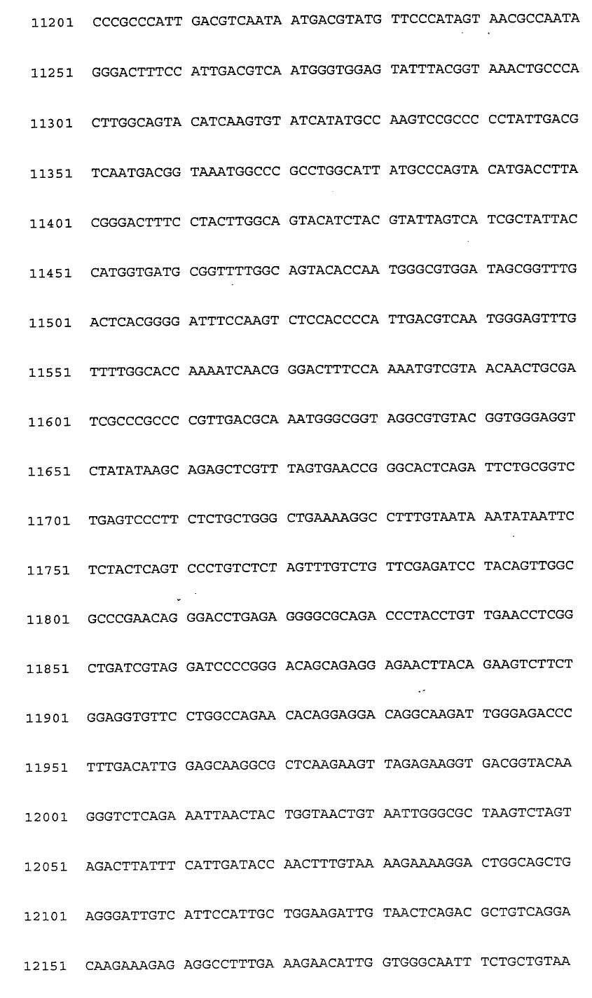

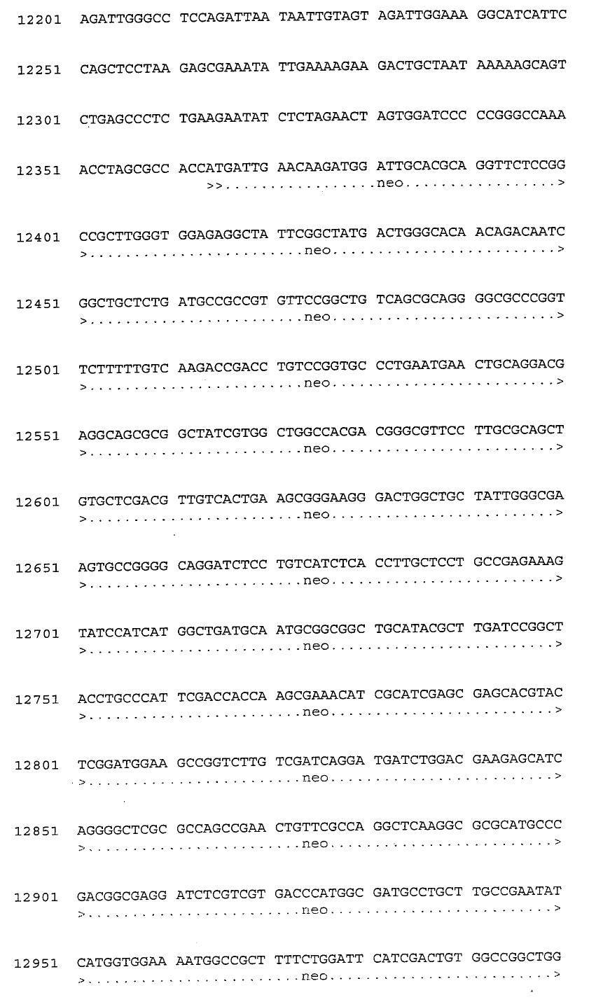

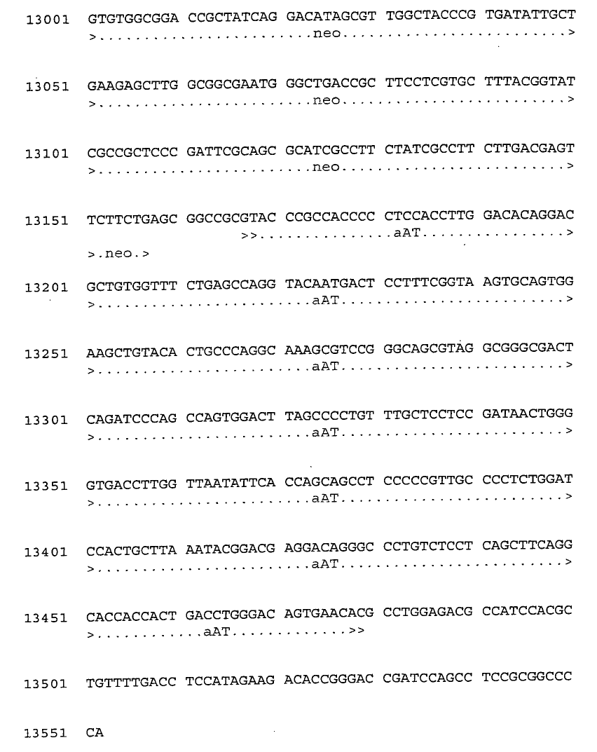

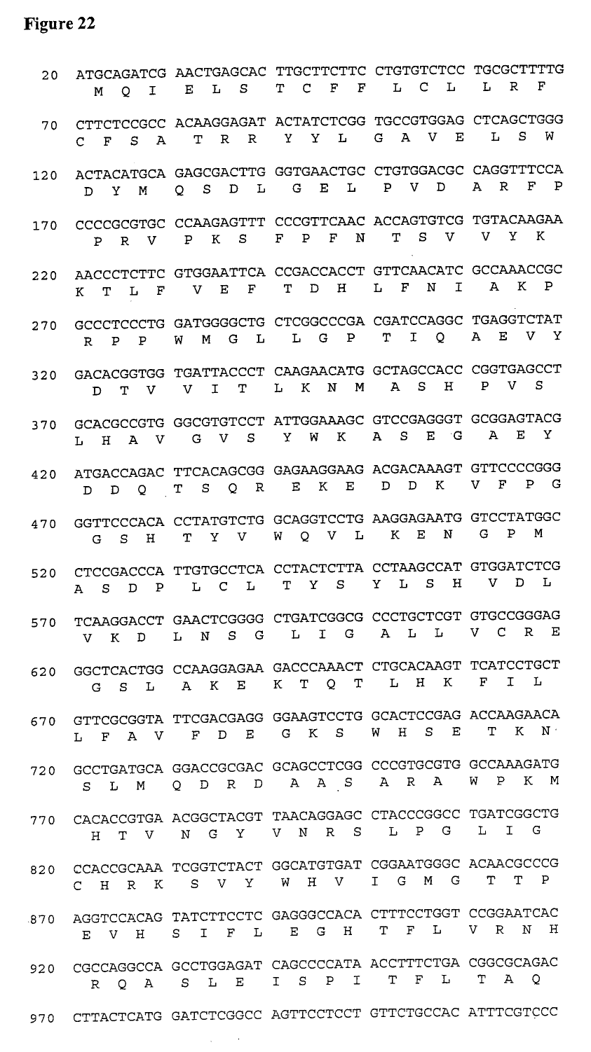

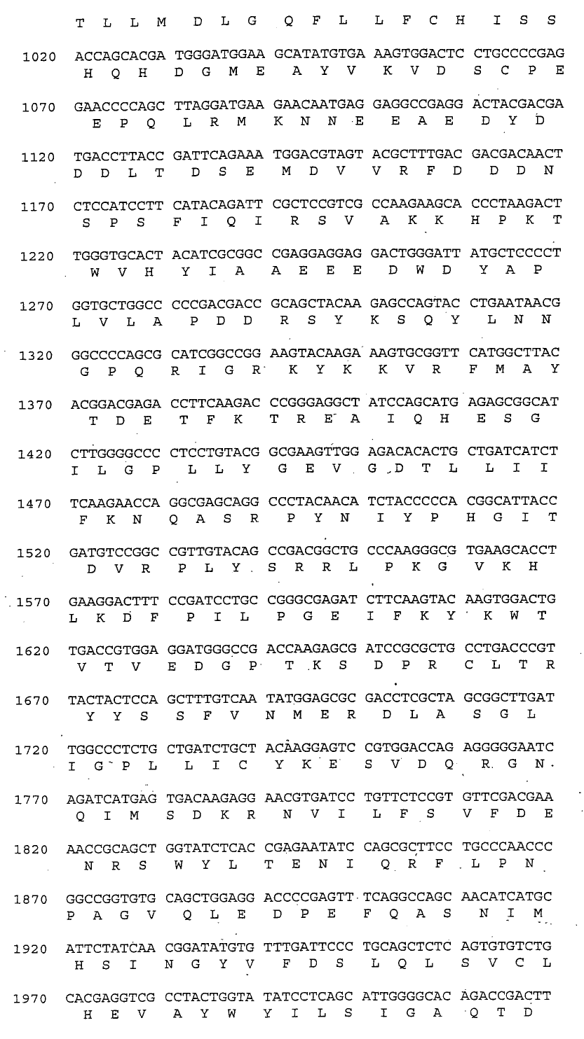

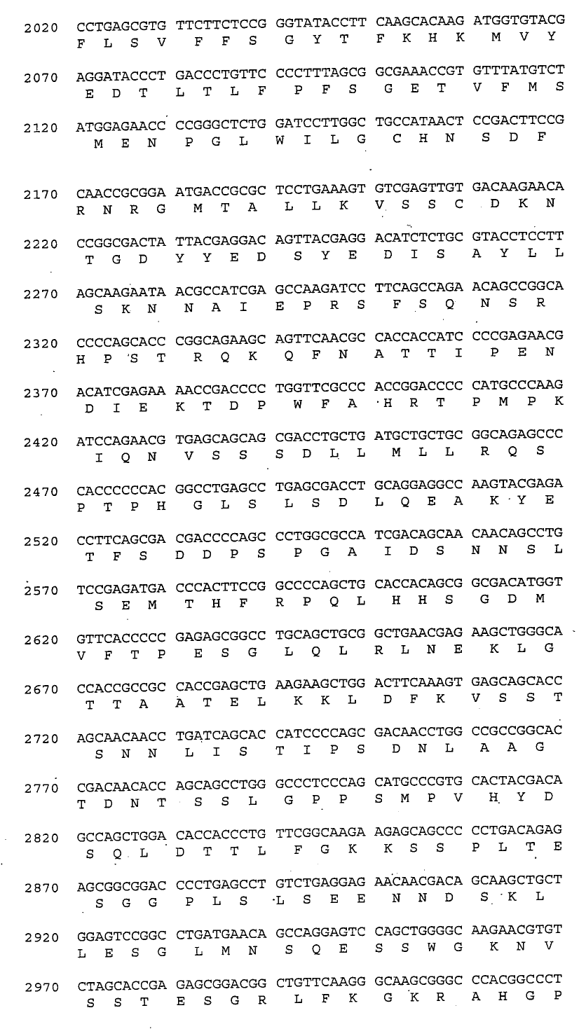

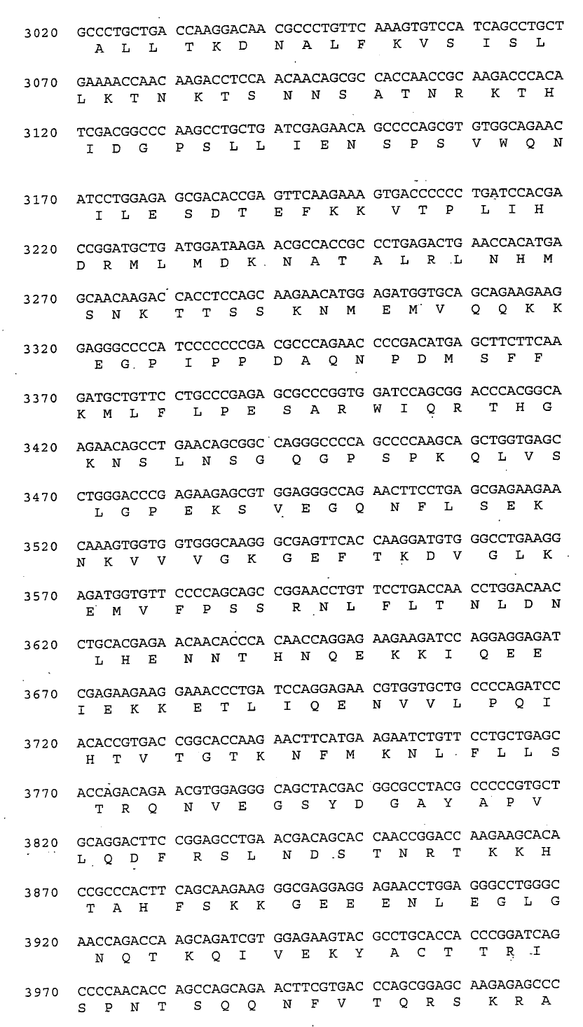

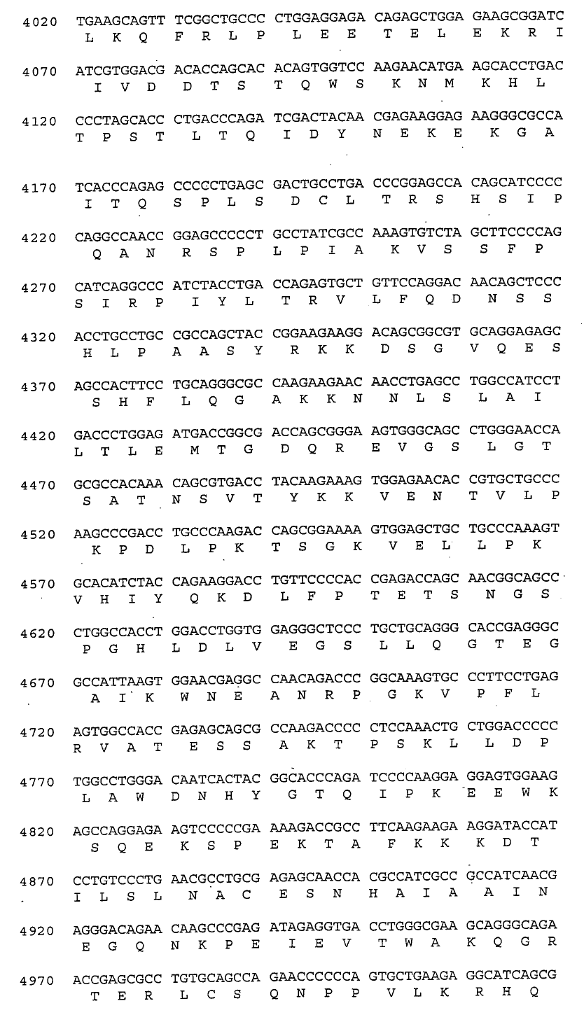

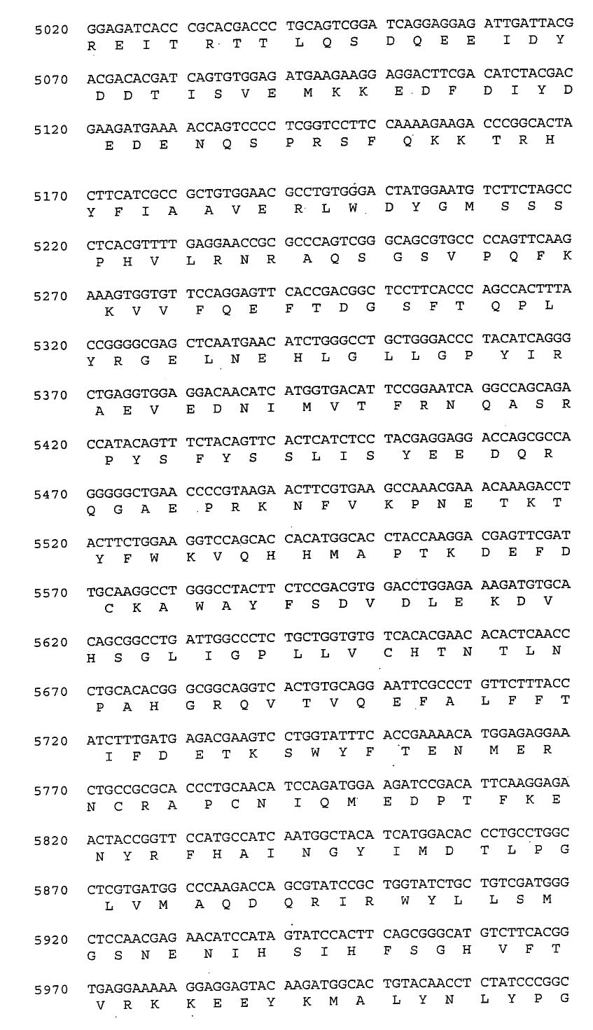

Examples of codon-optimised Factor VIII nucleotide sequences are shown in FIG. 19 and FIG. 21 (see bases 20 to 7072).

Construction of Genomes With Tissue Specific Promoters

Liver Specific Promoters

The human α₁-antitrypsin (hAAT) promoter is regarded as a strong liver-specific promoter. In a recent study the albumin, human α₁-antitrypsin and hemopexin promoters (alone and combined with enhancer regions) were tested in vitro and in mice by hydrodynamic delivery (Kramer et al 2003 ibid). In vivo data from a long term study (50d) showed that the human α₁-antitrypsin promoter resulted in stable levels of reporter gene expression. In an earlier study in which the hAAT, murine albumin, rat phosphoenolpyruvate carboxykinase (PEPCK) and rat liver fatty acid binding protein promoters were compared in the context of a retroviral vector, the hAAT promoter was found to result in the highest expression (Hafenrichter et al 1994 Blood 84: 3394-404). However use may be made of any of the aforementioned liver promoters.

The hAAT promoter was selected for testing. The promoter was cloned by PCR from HT1080 genomic DNA using primers based on those described in Kramer et al 2003 ibid with some modifications. The primers used are:

(including restriction sites & overhangs):

| | |
|---|---|
| HAATN: | |
| TATGAGCGGCCGCGTACCCGCCACCCCCTCCACCTTG | (SEQ ID NO:1) |
| G (contains NotI site) | |
| HAATP: | |
| ATCATGCACGTGTTCACTGTCCCAGGTCAGTGGTG | (SEQ ID NO:2) |
| (contains PmlI site) | |

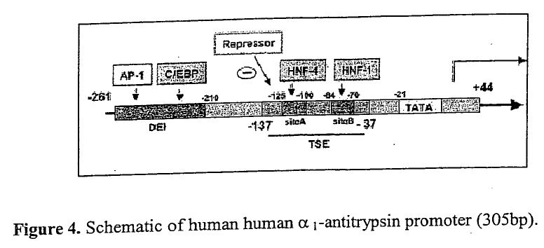

A schematic of the promoter is shown in FIG. 4.

Use may also be made of liver-specific enhancer elements such as human serum albumin enhancers, human prothrombin enhancers, α-1 microglobulin enhancers and intronic aldolase enhancers. The tissue specific promoter used in the present invention may include one or more enhancers, such as, but not limited to, the hepatic locus control region from the apolipoprotein E (ApoE) gene (HCR), the hepatitis B virus (HBV) enhancer 2 element and the albumin enhancer.

Endothelial Specific Promoters

A number of publications describe analysis of endothelial specific promoters which may be used in the invention including fins-like tyrosine kinase-1 (Flt-1/VEGF receptor-1), intercellular adhesion molecule-2 (ICAM-2), von Willebrand Factor (vWF), VEGF receptor-2 (Flk-1/KDR), endoglin (Nicklin et al 2001 Hypertension 38: 65-70; Kappel et al 1999 Blood 93:4284-92; Cowan et al 1998 J. Biol. Chem. 273: 11737-44; Velasco et al 2001 Gene Ther. 8:897-904) and the tie promoters, such as tie 1 and tie 2 (Korhonen et al 1 Blood 86:1828-35).

The ICAM-2 promoter may be amplified from 293T genomic DNA using primers based on those described in Nicklin et al 2001 ibid.

Prevention of Transgene Expression in Producer Cells

In a highly preferred embodiment, a B-domain deleted Factor VIII gene was inserted into a vector of the first aspect of the present invention, under the control of the human alpha one antitrypsin (hAAT) liver specific promoter. This allowed for the vector to be produced in high enough titres to be used in gene therapy to alleviate haemophilia. Circumventing the problem of vector production caused by expression of Factor VIII within the producer cells.

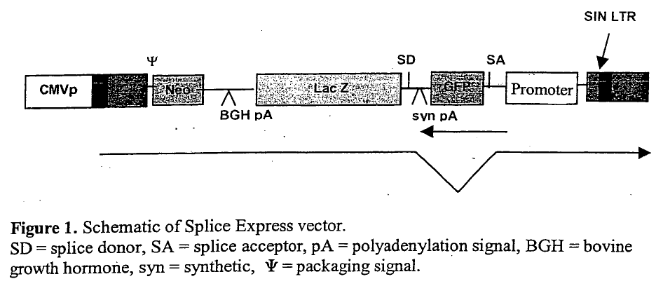

As the expression of Factor VIII in producer cells appears to reduce titres an alternative strategy for preventing expression in these cells was devised. The strategy exploits the ability to produce new generation EIAV vectors in the absence of Rev. An open reading frame (ORF) is inserted between the internal promoter and the therapeutic gene, all of which are in the reverse orientation. Therefore the protein encoded by this ORF, and not the therapeutic, will be expressed in the producers. The ORF, and its polyadenylation signal, are contained within an intron such that (in the absence of Rev) it will be spliced out of the genome transcript prior to packaging. This is shown in FIG. 1.

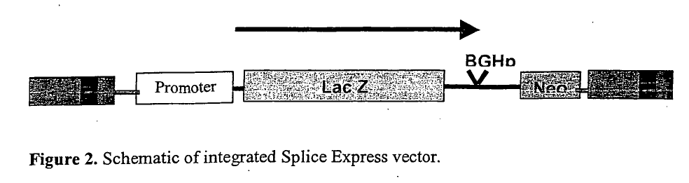

As the first ORF has been spliced out of the genome transcript, the therapeutic gene will be expressed in the transduced cells following integration (FIG. 2).

To test the strategy a vector containing LacZ and GFP reporter genes, as depicted in FIG. 1 was constructed. By using these vectors LacZ protein expression is minimal in producer cells yet high level expression is attained upon transduction.

Retroviruses

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host cell for the purpose of replicating the vectors comprising a segment of DNA. Examples of vectors used in recombinant DNA techniques include but are not limited to plasmids, chromosomes, artificial chromosomes or viruses.

The term “expression vector” means a construct capable of in vivo or in vitro/ex vivo expression.

The retroviral vector employed in the aspects of the present invention may be derived from or may be derivable from any suitable retrovirus. A large number of different retroviruses have been identified. Examples include: murine leukemia virus (MLV), human immunodeficiency virus (HIV), human T-cell leukemia virus (HTLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney munrine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “retroviruses”, Cold Spring Harbour Laboratory Press Eds: J M Coffin, S M Hughes, H E Varmus pp 758-763.

Retroviruses may be broadly divided into two categories: namely, “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al., 1997 (ibid).

In a typical vector for use in the method of the present invention, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target non-dividing host cell and/or integrating its genome into a host genome.

Preferably the viral vector capable of transducing a target non-dividing or slowly dividing cell is a lentiviral vector.

Lentivirus vectors are part of a larger group of retroviral vectors. A detailed list of lentiviruses may be found in Coffin et al (“Retroviruses” 1997 Cold Spring Harbour Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells (Lewis et a/1992 EMBO. J 11: 3053-3058; Lewis and Emerman 1994 J. Virol. 68: 510-516). In contrast, other retroviruses—such as MLV—are unable to infect non-dividing or slowly dividing cells such as those that make up, for example, muscle, brain, lung and liver tissue.

A “non-primate” vector, as used herein in some aspects of the present invention, refers to a vector derived from a virus which does not primarily infect primates, especially humans. Thus, non-primate virus vectors include vectors which infect non-primate mammals, such as dogs, sheep and horses, reptiles, birds and insects.

A lentiviral or lentivirus vector, as used herein, is a vector which comprises at least one component part derivable from a lentivirus. Preferably, that component part is involved in the biological mechanisms by which the vector infects cells, expresses genes or is replicated. The term “derivable” is used in its normal sense as meaning the sequence need not necessarily be obtained from a retrovirus but instead could be derived therefrom. By way of example, the sequence may be prepared synthetically or by use of recombinant DNA techniques.

The non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV). Preferably the lentivirus is an EIAV. Equine infectious anaemia virus infects all equidae resulting in plasma viremia and thrombocytopenia (Clabough, et al. 1991. J. Virol. 65:6242-51). Virus replication is thought to be controlled by the process of maturation of monocytes into macrophages.

In one embodiment the viral vector is derived from EIAV. EIAV has the simplest genomic structure of the lentiviruses and is particularly preferred for use in the present invention. In addition to the gag, pol and env genes EIAV encodes three other genes: tat, rev, and S2. Tat acts as a transcriptional activator of the viral LTR (Derse and Newbold 1993 Virology. 194:530-6; Maury, et al 1994 Virology. 200:632-42) and Rev regulates and coordinates the expression of viral genes through rev-response elements (RRE) (Martarano et al 1994 J. Virol. 68:3102-11). The mechanisms of action of these two proteins are thought to be broadly similar to the analogous mechanisms in the primate viruses (Martano et al ibid). The function of S2 is unknown. In addition, an EIAV protein, Ttm, has been identified that is encoded by the first exon of tat spliced to the env coding sequence at the start of the transmembrane protein.

In addition to protease, reverse transcriptase and integrase non-primate lentiviruses contain a fourth pol gene product which codes for a dUTPase. This may play a role in the ability of these lentiviruses to infect certain non-dividing cell types.

The viral RNA of this aspect of the invention is transcribed from a promoter, which may be of viral or non-viral origin, but which is capable of directing expression in a eukaryotic cell such as a mammalian cell. Optionally an enhancer is added, either upstream of the promoter or downstream. The RNA transcript is terminated at a polyadenylation site which may be the one provided in the lentiviral 3′ LTR or a different polyadenylation signal.

Thus the present invention employs a DNA transcription unit comprising a promoter and optionally an enhancer capable of directing expression of a non-primate lentiviral vector genome.

Transcription units as described herein comprise regions of nucleic acid containing sequences capable of being transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Nucleic acids may be, for example, ribonucleic acid (RNA) or deoxyribonucleic acid (DNA) or analogues thereof. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements. Nucleic acids may comprise cDNA or genomic DNA (which may contain introns).

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a defective retroviral vector genome gag, pol and env may be absent or not functional. The R regions at both ends of the RNA are repeated sequences. U5 and U3 represent unique sequences at the 5′ and 3′ ends of the RNA genome respectively.

Preferred vectors for use in accordance with one aspect of the present invention are recombinant non-primate lentiviral vectors.

The term “recombinant lentiviral vector” (RLV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RLV carries non-viral coding sequences which are to be delivered by the vector to the target cell. An RLV is incapable of independent replication to produce infectious retroviral particles within the final target cell. Usually the RLV lacks a functional gag-pol and/or env gene and/or other genes essential for replication. The vector of the present invention may be configured as a split-intron vector. A split intron vector is described in PCT patent application WO 99/15683.

Preferably the lentiviral vector of the present invention has a minimal viral genome.

As used herein, the term “minimal viral genome” means that the viral vector has been manipulated so as to remove the non-essential elements and to retain the essential elements in order to provide the required functionality to infect, transduce and deliver a nucleotide sequence of interest to a target host cell. Further details of this strategy can be found in our WO98/17815.

A minimal lentiviral genome for use in the present invention will therefore comprise (5′) R-U5—one or more first nucleotide sequences —U3-R (3′). However, the plasmid vector used to produce the lentiviral genome within a host cell/packaging cell will also include transcriptional regulatory control sequences operably linked to the lentiviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter. Some lentiviral genomes require additional sequences for efficient virus production. For example, in the case of HIV, rev and RRE sequence are preferably included. However the requirement for rev and RRE may be reduced or eliminated by codon optimisation. Further details of this strategy can be found in our WO01/79518. Alternative sequences which perform the same function as the rev/RRE system are also known. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. Any other functional equivalents which are known or become available may be relevant to the invention. For example, it is also known that the Rex protein of HTLV-1 can functionally replace the Rev protein of HIV-1. It is also known that Rev and Rex have similar effects to IRE-BP.

In one embodiment of the present invention, the lentiviral vector is a self-inactivating vector.

By way of example, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al 1986 Proc Natl Acad Sci 83: 3194-3198; Dougherty and Temin 1987 Proc Natl Acad Sci 84: 1197-1201; Hawley et al 1987 Proc Natl Acad Sci 84: 2406-2410; Yee et al 1987 Proc Natl Acad Sci 91: 9564-9568). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al 1983 Nucleic Acids Res 11: 1855-1872) or suppression of transcription (Emerman and Temin 1984 Cell 39: 449-467). This strategy can also be used to eliminate downstream transcription from the 3′ LTR into genomic DNA (Herman and Coffin 1987 Science 236: 845-848). This is of particular concern in human gene therapy where it is of critical importance to prevent the adventitious activation of an endogenous oncogene.

In our WO99/32646 we give details of features which may advantageously be applied to the present invention. In particular, it will be appreciated that the non-primate lentivirus genome (1) preferably comprises a deleted gag gene wherein the deletion in gag removes one or more nucleotides downstream of about nucleotide 350 or 354 of the gag coding sequence; (2) preferably has one or more accessory genes absent from the non-primate lentivirus genome; (3) preferably lacks the tat gene but includes the leader sequence between the end of the 5′ LTR and the ATG of gag; and (4) combinations of (1), (2) and (3). In a particularly preferred embodiment the lentiviral vector comprises all of features (1) and (2) and (3).

The non-primate lentiviral vector may be a targeted vector. The term “targeted vector” refers to a vector whose ability to infect/transfect/transduce a cell or to be expressed in a host and/or target cell is restricted to certain cell types within the host organism, usually cells having a common or similar phenotype.

Expression may be controlled using control sequences, which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters.

Suitable promoting sequences are strong promoters including those derived from the genomes of viruses—such as polyoma virus, adenovirus, fowlpox virus, bovine papilloma virus, avian sarcoma virus, cytomegalovirus (CMV), retrovirus and Simian Virus 40 (SV40)- or from heterologous mammalian promoters—such as the actin promoter or ribosomal protein promoter. Transcription of a gene may be increased further by inserting an enhancer sequence into the vector. Enhancers are relatively orientation- and position-independent, however, one may employ an enhancer from a eukaryotic cell virus—such as the SV40 enhancer on the late side of the replication origin (bp 100-270) and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable host. For example, the features can be conserved regions e.g. a Pribnow Box or a TATA box. The promoter may even contain other sequences to affect (such as to maintain, enhance, decrease) the levels of expression of a nucleotide sequence. Suitable other sequences include the Sh1-intron or an ADH intron. Other sequences include inducible elements—such as temperature, chemical, light or stress inducible elements. Also, suitable elements to enhance transcription or translation may be present.

The expression vector of the present invention comprises a signal sequence and an amino-terminal tag sequence operably linked to a nucleotide sequence of interest.

In an especially preferred embodiment of the present invention, when the NOI encodes for Factor VIII a tissue specific promoter as discussed above is employed.

By using producer/packaging cell lines, it is possible to propagate and isolate quantities of retroviral vector particles (e.g. to prepare suitable titres of the retroviral vector particles) for subsequent transduction of, for example, a site of interest (such as adult brain tissue). Producer cell lines are usually better for large scale production or vector particles.

Transient transfection has numerous advantages over the packaging cell method. In this regard, transient transfection avoids the longer time required to generate stable vector-producing cell lines and is used if the vector genome or retroviral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produce vector titre levels that are comparable to the levels obtained from stable vector-producing cell lines (Pear et al 1993, PNAS 90:8392-8396).

Producer cells/packaging cells can be of any suitable cell type. Producer cells are generally mammalian cells but can be, for example, insect cells.

As used herein, the term “producer cell” or “vector producing cell” refers to a cell which contains all the elements necessary for production of retroviral vector particles.

Preferably, the producer cell is obtainable from a stable producer cell line.

Preferably, the producer cell is obtainable from a derived stable producer cell line.

Preferably, the producer cell is obtainable from a derived producer cell line.

As used herein, the term “derived producer cell line” is a transduced producer cell line which has been screened and selected for high expression of a marker gene. Such cell lines support high level expression from the retroviral genome. The term “derived producer cell line” is used interchangeably with the term “derived stable producer cell line” and the term “stable producer cell line.

Preferably the derived producer cell line includes but is not limited to a retroviral and/or a lentiviral producer cell.

Preferably the derived producer cell line is an HIV or EIAV producer cell line, more preferably an EIAV producer cell line.

Preferably the envelope protein sequences, and nucleocapsid sequences are all stably integrated in the producer and/or packaging cell. However, one or more of these sequences could also exist in episomal form and gene expression could occur from the episome.

As used herein, the term “packaging cell” refers to a cell which contains those elements necessary for production of infectious recombinant virus which are lacking in the RNA genome. Typically, such packaging cells contain one or more producer plasmids which are capable of expressing viral structural proteins (such as codon optimised gag-pol and env) but they do not contain a packaging signal.

The term “packaging signal” which is referred to interchangeably as “packaging sequence” or “psi” is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon.

Packaging cell lines suitable for use with the above-described vector constructs may be readily prepared (see also WO 92/05266), and utilised to create producer cell lines for the production of retroviral vector particles. As already mentioned, a summary of the available packaging lines is presented in “Retroviruses” (as above).

Also as discussed above, simple packaging cell lines, comprising a provirus in which the packaging signal has been deleted, have been found to lead to the rapid production of undesirable replication competent viruses through recombination. In order to improve safety, second generation cell lines have been produced wherein the 3′LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection.

Preferably, the packaging cell lines are second generation packaging cell lines.

Preferably, the packaging cell lines are third generation packaging cell lines.

In these split-construct, third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre vector particle production. The packaging cell may be a cell cultured in vitro such as a tissue culture cell line. Suitable cell lines include but are not limited to mammalian cells such as munrine fibroblast derived cell lines or human cell lines. Preferably the packaging cell line is a human cell line, such as for example: HEK293, 293-T, TE671, HT1080.

Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

In more detail, the packaging cell may be an in vivo packaging cell in the body of an individual to be treated or it may be a cell cultured in vitro such as a tissue culture cell line. Suitable cell lines include mammalian cells such as murine fibroblast derived cell lines or human cell lines. Preferably the packaging cell line is a human cell line, such as for example: 293 cell line, HEK293, 293-T, TE671, HT1080.

Alternatively, the packaging cell may be a cell derived from the individual to be treated such as a monocyte, macrophage, stem cells, blood cell or fibroblast. The cell may be isolated from an individual and the packaging and vector components administered ex vivo followed by re-administration of the autologous packaging cells. Alternatively the packaging and vector components may be administered to the packaging cell in vivo. Methods for introducing lentiviral packaging and vector components into cells of an individual are known in the art. For example, one approach is to introduce the different DNA sequences that are required to produce a lentiviral vector particle e.g. the env coding sequence, the gag-pol coding sequence and the defective lentiviral genome into the cell simultaneously by transient triple transfection (Landau & Littman 1992 J. Virol. 66, 5110; Soneoka et al 1995 Nucleic Acids Res 23:628-633).

In one embodiment the vector configurations of the present invention use as their production system, three transcription units expressing a genome, the gag-pol components and an envelope. The envelope expression cassette may include one of a number of envelopes such as VSV-G or various murine retrovirus envelopes such as 4070A.

Conventionally these three cassettes would be expressed from three plasmids transiently transfected into an appropriate cell line such as 293T or from integrated copies in a stable producer cell line. An alternative approach is to use another virus as an expression system for the three cassettes, for example baculovirus or adenovirus. These are both nuclear expression systems. To date the use of a poxvirus to express all of the components of a lentiviral vector system has not been described. In particular, given the unusual codon usage of lentiviruses and their requirement for RNA handling systems such as the rev/RRE system

Pseudotyping

In one preferred aspect, the retroviral vector of the present invention has been pseudotyped. In this regard, pseudotyping can confer one or more advantages. For example, with the lentiviral vectors, the env gene product of the HIV based vectors would restrict these vectors to infecting only cells that express a protein called CD4. But if the env gene in these vectors has been substituted with env sequences from other RNA viruses, then they may have a broader infectious spectrum (Verma and Somia 1997 Nature 389:239-242). By way of example, workers have pseudotyped an HIV based vector with the glycoprotein from VSV (Verma and Somia 1997 ibid).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al 1996 Gene Therapy 3: 280-6; Fielding et al 1998 Blood 9: 1802 and references cited therein).

The vector may be pseudotyped with any molecule of choice.

VSV-G:

Efficient transduction of hepatocytes has been achieved in vivo (mice) with VSV-G pseudotyped lentiviral vectors following non-invasive intravenous injection (tail vein) in the absence of DNA cycling (Follenzi et al 2002; Pan et al 2002). It has been suggested that the apparent discrepancy between these data, in line with others (Pfeifer et al 2001), and the previous finding that efficient transduction of liver requires cell cycling (Park et al 2000b) is due to improved vector design, specifically the inclusion of the cPPT, and increased particle infectivity. However in one study the vector used (HR′cmvGFP) does not contain the cPPT element and transduction of liver was observed: 59% GFP positive cells 4d post-injection, falling to 1.3% after 40d (Pan et al 2002).

Ross River Virus

The Ross River viral envelope has been used to pseudotype a nonprimate lentiviral vector (FIV) and following systemic administration predominantly transduced the liver (Kang et al 2002). Efficiency was reported to be 20-fold greater than obtained with VSV-G pseudotyped vector, and caused less cytotoxicity as measured by serum levels of liver enzymes suggestive of hepatotoxicity.

Ross River Virus (RRV) is an alphavirus spread by mosquitoes which is endemic and epidemic in tropical and temperate regions of Australia. Antibody rates in normal populations in the temperate coastal zone tend to be low (6% to 15%) although sero-prevalence reaches 27 to 37% in the plains of the Murray Valley River system. In 1979 to 1980 RRV became epidemic in the Pacific Islands. The disease is not contagious between humans and is never fatal, the first symptom being joint pain with fatigue and lethargy in about half of patients (Fields Virology).

Baculovirus GP64

The baculovirus GP64 protein has been shown to be an attractive alternative to VSVG for viral vectors used in the large-scale production of high-titer virus required for clinical and commercial applications (Kumar M, Bradow B P, Zimmerberg J, Hum Gene Ther. 2003 Jan. 1;14(1):67-77). Compared with VSVG, GP64 vectors have a similar broad tropism and similar native titers. Because, GP64 expression does not kill cells, 293T-based cell lines constitutively expressing GP64 can be generated.

Alternative Envelopes

Other envelopes which give reasonable titre when used to pseudotype EIAV include Mokola, Rabies, Ebola and LCMV (lymphocytic choriomeningitis virus). Following in utero injection in mice the VSV-G envelope was found to be more efficient at transducing hepatocytes than either Ebola or Mokola (Mackenzie et al 2002). Intravenous infusion into mice of lentivirus pseudotyped with 4070A led to maximal gene expression in the liver (Peng et al 2001.

Disruption of Tat

Disruption of the open reading frame of Tat enhances the safety profile of the vectors with no detrimental effect on titre despite the fact that the first exon of Tat is within the packaging signal.

This disruption may be achieved by the insertion of a nucleotide within the initial codon of the Tat open reading frame (plasmid nucleotides 1317-1319) in the vector genome.

gttgaacCTG->gttgaacCTCG (SEQ ID NOs:3 and 4, respectively)

This was confirmed by sequencing and titering of the new genome revealed no loss of titre resulting from this modification. Genomes without this modification express the amino-terminal portion (29 aa) of the viral protein Tat in the producer cells.

Mutation of Major Splice Donor (SD1)

We have found that the titre of vectors with this modification is at least as high as those with a functional major splice donor.

The disruption may be achieved by site-directed mutagenesis substituting nucleotide 1405 (T) for ‘C’ thereby destroying the splice donor.

AGGT->AGGC

The mutated splice donor is non-functional as tested by insertion of a functional splice acceptor downstream.

Inclusion of WPRE/cPPT Elements

The WPRE element enhances expression and as such is likely to be beneficial in attaining maximal levels of Factor VIII.

Transgene Expression in Producer Cells

In order to minimise potential for expression of the transgene in producer cells, such as 293T cells, the cloning of transgenes into the vectors has been designed in such a way that the first NOI is out of frame with respect to any upstream ORFs.

Delivery Systems

The vector of the present invention may be a delivered to a target site by a viral or a non-viral vector.

As it is well known in the art, a vector is a tool that allows or facilitates the transfer of an entity from one environment to another. By way of example, some vectors used in recombinant DNA techniques allow entities, such as a segment of DNA (such as a heterologous DNA segment, such as a heterologous cDNA segment), to be transferred into a target cell. Optionally, once within the target cell, the vector may then serve to maintain the heterologous DNA within the cell or may act as a unit of DNA replication. Examples of vectors used in recombinant DNA techniques include plasmids, chromosomes, artificial chromosomes or viruses.

Non-viral delivery systems include but are not limited to DNA transfection methods. Here, transfection includes a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

Viral delivery systems include but are not limited to adenovirus vector, an adeno-associated viral (AAV) vector, a herpes viral vector, retroviral vector, lentiviral vector, baculoviral vector. Other examples of vectors include ex vivo delivery systems, which include but are not limited to DNA transfection methods such as electroporation, DNA biolistics, lipid-mediated transfection, compacted DNA-mediated transfection.

The vector delivery system of the present invention may consist of a primary vector manufactured in vitro which encodes the genes necessary to produce a secondary vector in vivo.

The primary viral vector or vectors may be a variety of different viral vectors, such as retroviral, adenoviral, herpes virus or pox virus vectors, or in the case of multiple primary viral vectors, they may be a mixture of vectors of different viral origin. In whichever case, the primary viral vectors are preferably defective in that they are incapable of independent replication. Thus, they are capable of entering a target cell and delivering the secondary vector sequences, but not of replicating so as to go on to infect further target cells.

The delivery of one or more therapeutic genes by a vector system according to the present invention may be used alone or in combination with other treatments or components of the treatment.

For example, the retroviral vector of the present invention may be used to deliver one or more NOI(s) useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the retroviral vector of the present invention may be used to deliver one or more NOI(s) useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the retroviral vector of the present invention may be used to deliver one or more NOI(s) useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is particularly useful in the treatment of haemophilia.

The present invention also provides a pharmaceutical composition for treating an individual by gene therapy, wherein the composition comprises a therapeutically effective amount of the retroviral vector of the present invention comprising one or more deliverable therapeutic and/or diagnostic NOI(s) or a viral particle produced by or obtained from same. The pharmaceutical composition may be for human or animal usage. Typically, a physician will determine the actual dosage which will be most suitable for an individual subject and it will vary with the age, weight and response of the particular individual.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavemosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

In Vitro Production of Factor VIII

The vector or the nucleic acid encoding codon optimised Factor VIII of the present invention may also be used in the expression of Factor VIII in an in vitro/cell culture expression system. Accordingly, in another aspect of the invention, there is provided a host cell transduced with a vector or transfected with nucleic acid in accordance with any aspect of the invention.

Suitable host cells for transduction with a vector or nucleic acid encoding codon optimised Factor VIII of the invention include cells of a host organism, normal primary cells or cell lines derived from cultured primary tissue may be used. Suitably, cells are mammalian cells preferably hamster CHO cells, mouse C127 cells or human “293” cells. In another embodiment, the cells may be HepG2 cells as described herein.

Transduction of host cells involves incubating the vector or nucleic acid of the present invention with the host cell. Following passage of the transduced/transfected cells, media is removed for testing for Factor VIII activity using, for example, the COATEST (Chromogenix) as described herein.

Once the gene has been introduced into the suitable host cell, the host cell may be grown to high density in appropriate medium. The expressed Factor VIII can be extracted from the media of cells using conventional means, if secreted or isolated from cells using lysis. The desired product is then isolated and purified by conventional techniques, for example, affinity chromatography with immobilised antibodies, chromatography on aminohexyl-sepharose or the mixed polyelectrolyte method.

Accordingly, in a further aspect of the invention there is provided a method for producing Factor VIII in vitro comprising generating a cell in accordance with the invention, passaging said cell in media, removing said media and isolating Factor VIII.

In another aspect of the invention, there is provided a method for producing Factor VIII in vitro comprising generating a cell comprising a codon optimised nucleic acid encoding Factor VIII in accordance with the invention, passaging said cell in media, removing said media and isolating Factor VIII.

### EXAMPLES

Vector Construction

Details of pONY8.4 can be found in our WO03/064665. In more detail, pONY 8.4 series of vectors has a number of modifications which enable it to function as part of a transient or stable vector system totally independent of accessory proteins, with no detrimental effect on titre. Conventionally lentiviral vector genomes have required the presence of the viral protein rev in producer cells (transient or stable) in order to obtain adequate titres. This includes current HIV vector systems as well as earlier EIAV vectors.

There are 4 modifications when compared with the pONY 8.1 series of vector genomes, these are:
a) All the ATG motifs which are derived from gag and form part of the packaging signal have been modified to read ATTG. This allows the insertion of an open reading frame which can be driven by a promoter in the LTR.
b) The length of the genome i.e. distance between the R regions is closer to that seen in the wt virus (7.9 kb).
c) The 3′ U3 region has been modified to include sequences from the Moloney leukemia virus (MLV) U3 region, so upon transduction it can drive second open reading frame (ORF) in addition to the internal cassette, In this example we have MLV but this could be any promoter.
d) The vector contains a nucleotide sequence operably linked to the viral LTR and wherein said nucleotide sequence is upstream of an internal promoter and wherein said nucleotide sequence encodes a polypeptide or fragment thereof.

Together these modifications allow production of viral delivery system without the need for accessory proteins and only 10% of the original viral sequence is integrated into the target cell. These factors are important for future safety considerations in terms of an immune response and probability of the generation of replication competent viruses. Further details on modifying LTRs can be found in our WO96/37623 and WO98/17816.

pONY8.7 series vectors have cPPT and WPRE (pONY8.4 have neither).

pONY8.8 series vectors have cPPT but no WPRE.

pONY8.9 series vectors have WPRE but no cPPT.

In the vectors the suffix 5 (e.g. pONY8.95) indicates both Tat and splice donor modifications as described below.

In the vectors the suffix 3 (e.g. pONY8.43) indicates both Tat but not splice donor modifications as described below.

In the vector nomenclature:

“N” indicates the presence of neo,

“C” indicates the presence of CMV,

“G” indicates the presence of GFP,

“F” or “HEN” or “HENSQ” indicates the presence of the codon-optimised B domain deleted Factor VIII,

“Z” indicates the presence of LacZ,

“A” indicates the presence of hAAT,

“I” indicates the presence of ICAM-2.

So, by way of illustration: pONY8.4NCZ has a SIN LTR, neo is not expressed, upstream ORF for Rev independence. pONY8.95NCZ has WPRE, no cPPT, a SIN LTR so neo is not expressed, and the Tat Exon 1 and SD1 are mutated. pONY8.7NCF has cPPT, WPRE, the upstream ORF is neo, a CMV internal promoter, codon-optimised B domain deleted Factor VIII.

Analysis of Vectors

Predicted Titre by PERT (Performance Enhanced Reverse Transcription)

Vector genomes expressing LacZ or Factor VIII from an internal CMV promoter were used to prepare vector pseudotyped with VSV-G. Real time PCR was used to quantitate reverse transcriptase activity by measurement of RT-PCR products from MS2 RNA template following particle disruption. The predicted number of vector particles (titre) is determined by comparing unknowns with a reference standard.

Predicted titres of the Factor VIII genomes were lower than those for Lac Z, although the difference was within 1 log.

Titre by RNA Genome Level

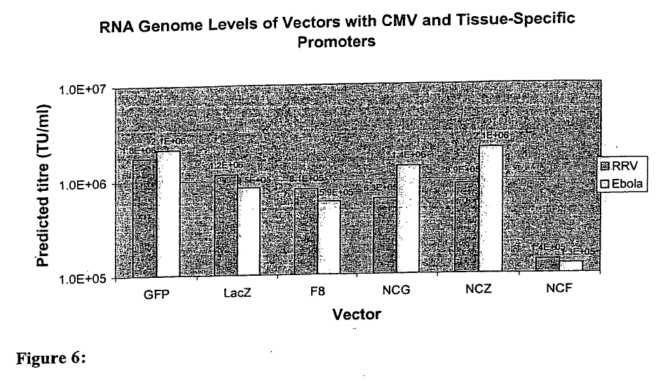

Vector genomes expressing the GFP, LacZ and Factor VIII transgenes from the CMV or tissue-specific promoters were used to prepare viral vector. Vectors containing the hAAT internal promoter were pseudotyped with the Ross River Virus (RRV) envelope and those with the ICAM-2 promoter were pseudotyped with the Ebola envelope. The selection of envelope was based on the target cell type: the Ebola envelope permits efficient transduction of HUVEC cells selected for testing the activity of the ICAM-2 promoter and the RRV envelope has been reported to enable efficient transduction of hepatic cells (Kang et al 2002). Control vectors containing the internal CMV promoter were pseudotyped with both envelopes. Results from real-time PCR analysis of viral RNA levels are shown in FIG. 6.

Predicted titres of the Factor VIII genomes containing a tissue-specific internal promoter are around five-fold higher than titres obtained with the standard CMV (which consistently gives a predicted titre of 1×10⁵ TU/ml).

Promoter Activity in 293T Cells

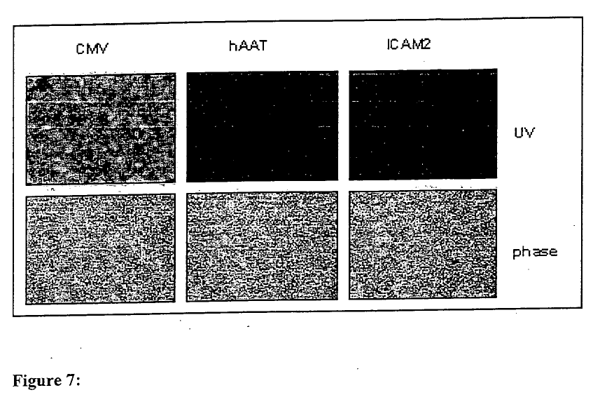

In order to determine the relative activities of the ICAM-2, hAAT and CMV promoters in producer cells, 293Ts were transiently transfected with genomes expressing GFP. Cells were viewed by UV microscope approximately 24 h post sodium butyrate treatment, 36 h post-transfection. Representative images are shown in FIG. 7.

Promoter Activity in Target Cells

Liver Cells

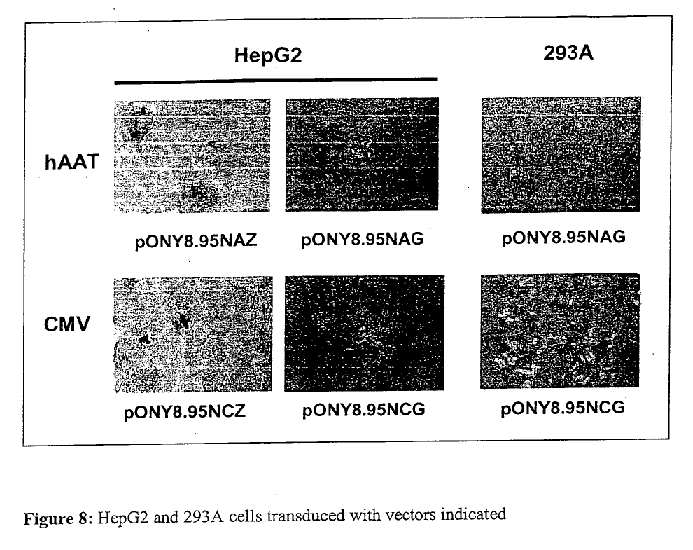

The human hepatocellular carcinoma cell line, Hep G2, was selected for testing the activity of the hAAT promoter. This was previously used for in vitro testing of this promoter (Kramer et al 2003) which was reported to have an activity 40% of that of the immediate-early cytomegalovirus (CMV) promoter (including enhancer regions). Representative images of HepG2 and 293A cells transduced with vectors expressing reporter genes from either the CMV or hAAT promoters are shown in FIG. 8.

Using both β-galactosidase and GFP reporter genes, colonies of transduced cells were easily visualised when either CMV or hAAT promoters were used to drive transgene expression. Biological titres (X-gal stained cells) were equivalent reflecting the comparable titre as measured by RNA genome levels and indicating activity of the two promoters is similar in HepG2s. This was supported by β-galactosidase assay of lysates prepared from transduced cells.

Endothelial Cells

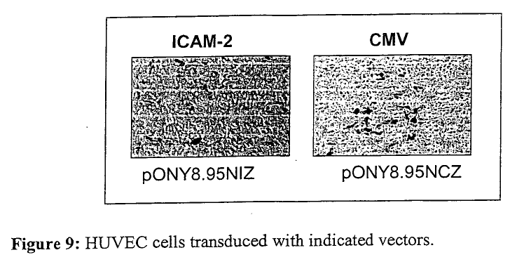

HUVECs (human embilical vein endothelial cells) were selected for testing the activity of the ICAM-2 promoter. Images of X-gal stained cells transduced with vectors expressing LacZ from the ICAM-2 and CMV promoters are shown in FIG. 9.

293A Cells

FACS analysis showed no GFP positive cells could be detected in 293A cells transduced with the vectors containing tissue-specific promoters. This is in contrast with CMV control vectors which resulted in populations of highly expressing cells.

In summary, both tissue-specific promoters, ICAM-2 and hAAT, resulted in low levels of activity in 293 (293A and 293T) cells as desired. Evidence of promoter activity could be detected in endothelial cells in the case of the ICAM-2 vector. In the case of the hAAT promoter very high activity was apparent in hepatic cells (comparable to the CMV promoter).

The low titre of vectors encoding Factor VIII expressed from a ubiquitous promoter is ascribed to expression of Factor VIII protein in 293T producer cells inhibiting the production of functional viral particles. Therefore strategies for avoiding transgene expression in 293Ts were sought. The most effective means of achieving this, whilst maintaining high transgene expression in target cells, has been replacing the internal CMV promoter with that of the strong liver specific human α₁-antitrypsin (hAAT) promoter. Additionally further improvements have been made to the genomes: mutation of the Tat exon 1 and of the major splice donor have been carried out without subsequent loss in titre.

Titre by Integration Assay

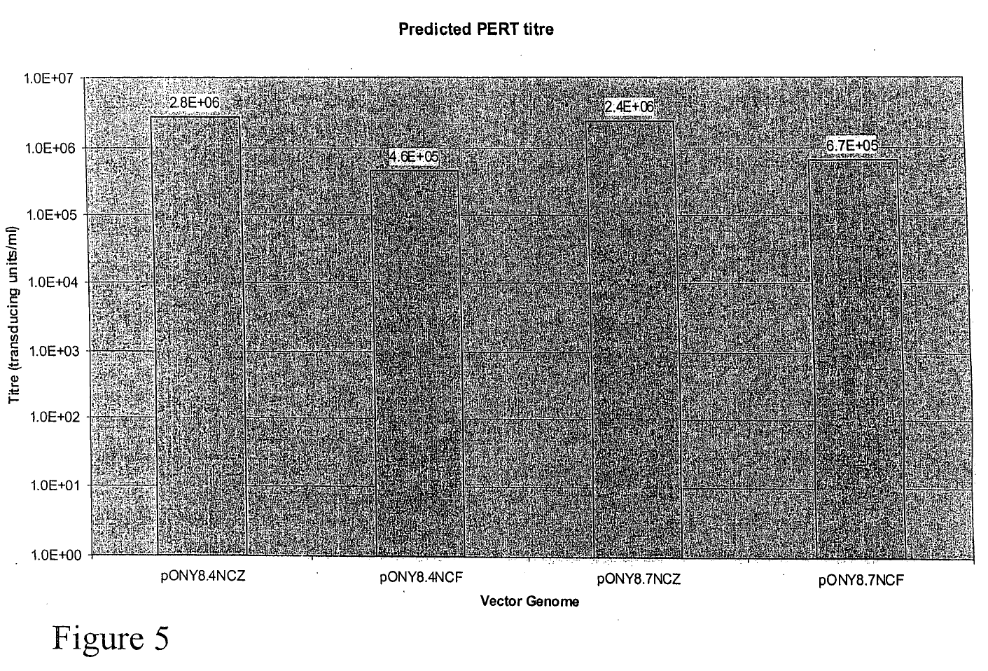

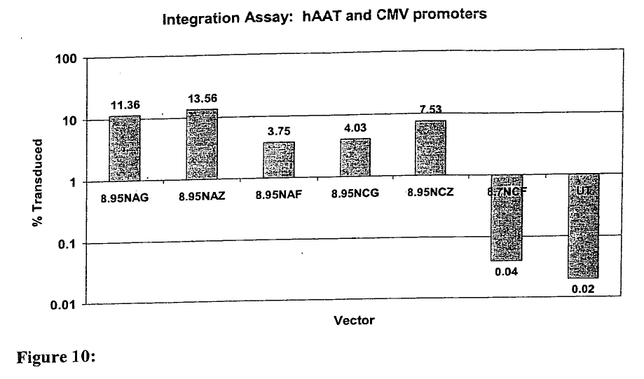

A functional assay of vector performance is critical to ascertain whether high titre vectors for the delivery of Factor VIII can be produced. As shown in FIGS. 5 and 6, neither RNA genome levels nor viral particle number (PERT) measurements are adequate for predicting titre. Therefore an integration assay was carried out by transducing 293A cells with viral supernatants. Data for the hAAT vectors, and CMV control vectors are shown in FIG. 10.

Cells transduced with pONY8.95NAF (Factor VIII expressed from hAAT promoter) contain similar levels of vector as those transduced with vector encoding a reporter gene (pONY8.95NCG). Cells transduced with pONY8.7NCF (internal CMV promoter), however, contain very low amounts of vector only slightly above background (UT=untransduced cells) reflecting the low functional titre obtained with this vector construct. These data indicate that the inhibition of particle production resulting from Factor VIII expression in producer cells has been completely circumvented by exchanging the CMV promoter for the hAAT promoter.

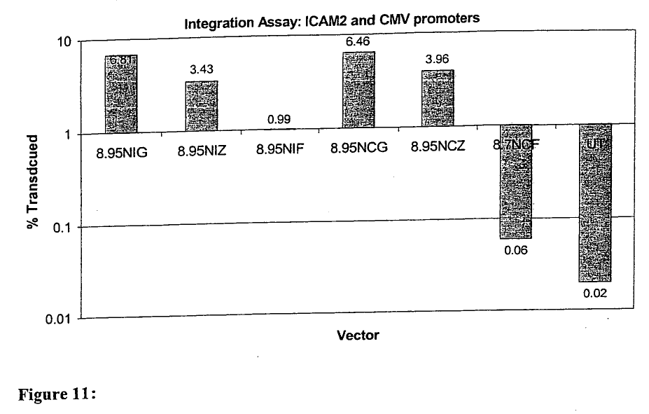

Data for the ICAM-2 vectors, and CMV control vectors are shown in FIG. 11.

As with the hAAT vector, use of the ICAM-2 promoter enables the production of Factor VIII vectors with high functional titre (approximately one third of LacZ control vectors).

Genome Mixing Experiments

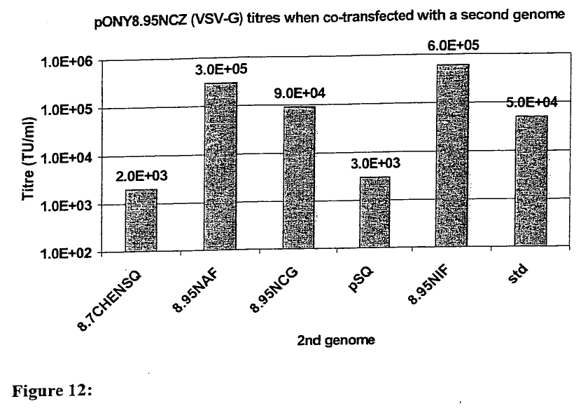

Co-transfection of a Factor VIII expressing genome (pONY8.7NCHENSQ), or a plasmid expressing Factor VIII (pSQ) routinely results in the decrease in titre of a vector expressing a reporter gene of around 2 logs. To confirm that co-transfection of the new Factor VIII genomes did not result in a disproportionate drop in titre of a second genome, they were co-transfected with pONY8.95NCZ and LacZ titres scored following titering on D17 cells. Results are shown in FIG. 12.

These data confirm the results of the integration assay: the new Factor VIII vector genomes do not cause inhibition of functional viral particle production.

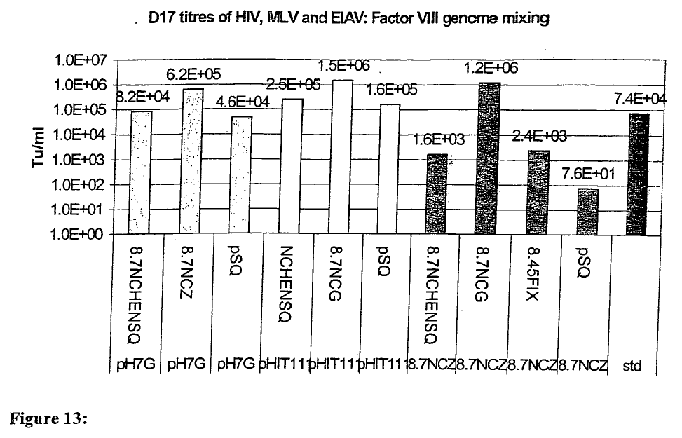

To ascertain whether the expression of Factor VIII protein in producer cells has an impact on functional titres of other lentiviral and retroviral vectors, the mixing experiment was conducted with HIV and MLV vectors. Data is shown in FIG. 13.

The data show a decrease in titre of approximately 1 log of MLV and HIV vectors when a plasmid expressing Factor VIII is included in the transfection. These data are in agreement with a similar previous experiment. Expression of Factor VIII in producer cells clearly has a detrimental effect on HIV and MLV vector titre although this is not as dramatic as with EIAV.

Construction of pONY8.45NCZ

Tat Exon1

Mutation of Tat exon1 was carried out by inserting a cytosine residue after nucleotide 434 (accession number EIU01866).

The oligonucleotides shown below were treated with T4 polynucleotide kinase using standard procedures, annealed then ligated into pONY8.4NCZ digested BseRI and Eco0109I (9463 bp fragment) to make pONY8.43NCZ.

Oligos used to mutate Exon1 of TAT:

| | |
|---|---|
| Oligo 1 | |
| GGGACCTGAGAGGGGCGCAGACCCTACCTGTTGAACC | (SEQ ID NO:5) |
| TCGGCTGATCGTAGGATCCCCGGGA | |
| Oligo 2 | |
| TGTAAGTTCTCCTCTGCTGTCCCGGGGATCCTACGAT | (SEQ ID NO:6) |
| CAGCCGAGGTTCAACAGGTAGGG | |

Major Splice Donor

Mutation of the major splice donor was achieved by exchanging the invariant tyrosine to cytosine using the following oligonucleotides:

| | |
|---|---|
| SD1KO1F: | |
| CAGAACACAGGAGGACAGGCAAGATTGGGAGACCCTT | (SEQ ID NO:7) |
| TG | |
| SD1KO2R: | |
| CAAAGGGTCTCCCAATCTTGCCTGTCCTCCTGTGTTC | (SEQ ID NO:8) |
| TG | |

(Altered nucleotide in bold).

The splice donor mutation was made using the QuickChange™ Site-Directed Mutagenesis kit from Stratagene and confirmed by sequencing. The construct containing both Tat exon 1 and major splice donor mutations was designated pONY8.45NCZ.

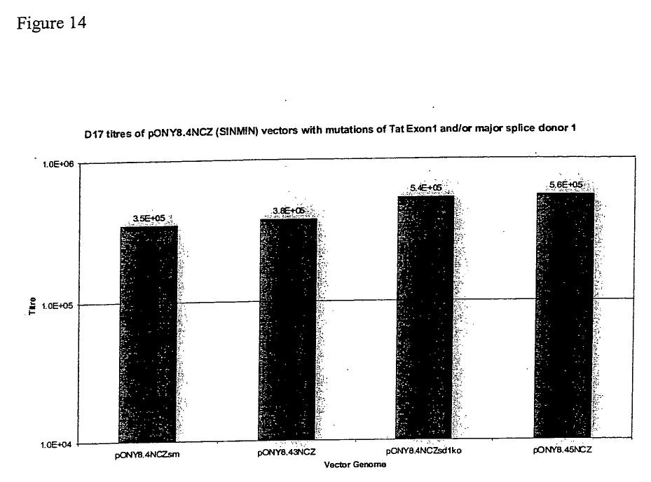

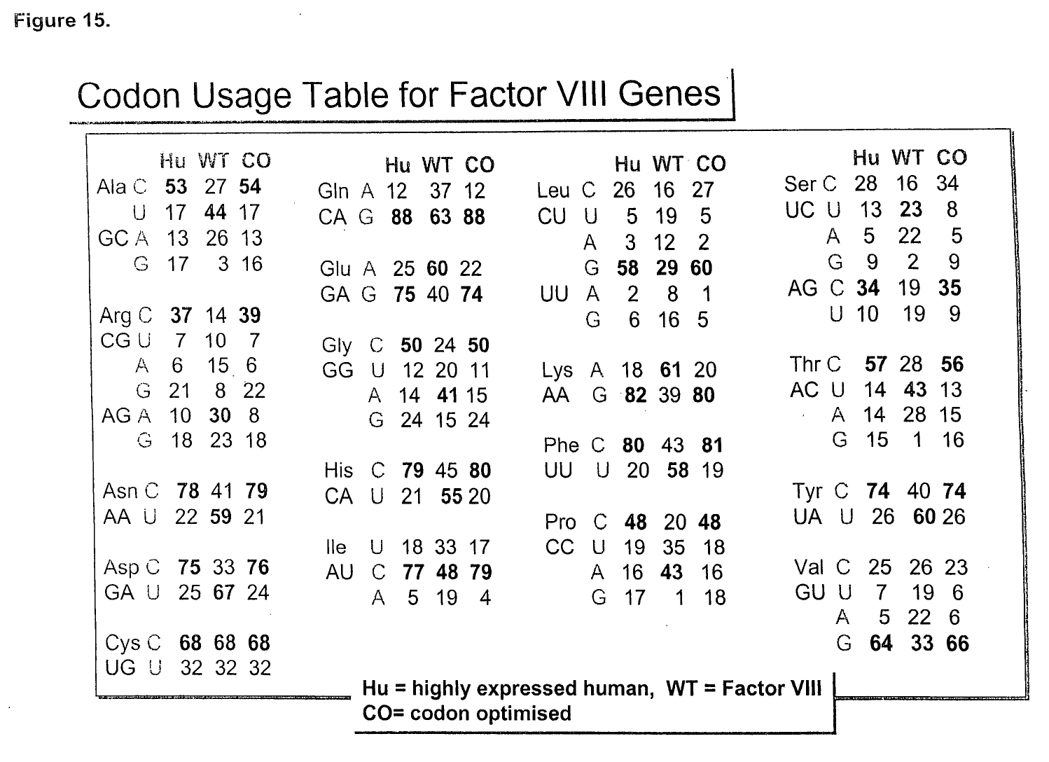

Neither single mutation, nor the two combined significantly altered titre. See data from first experiment in FIG. 14.

Titres of vectors containing the major splice donor were slightly enhanced. This has also been observed in subsequent experiments.

The following show mutations and insertions in the first exon of TAT, the major splice donor knock out and packaging signal of pONY 8.45NCZ vector.

| | |
|---|---|
| UI01866 401 | |
| cctgagaggggcgcagaccctacctgttgaacct-g | (SEQ ID NO:9) |
| gctgatcgtaggatccccgggacagcagaggagaac | |
| ttacagaagtcttctggaggtgttcctggccagaac | |
| acaggaggacag | |
| 8.45 NCZ 213 | |
| cctgagaggggcgcagaccctacctgttgaacctcg | (SEQ ID NO:10) |
| gctgatcgtaggatccccgggacagcagaggagaac | |
| ttacagaagtcttctggaggtgttcctggccagaac | |
| acaggaggacag | |
| UI01866 520 | |
| gtaagat-gggagaccctttgacat-ggagcaaggc | (SEQ ID NO:11) |
| gctcaagaagttagagaaggtgacggtacaagggtc | |
| tcagaaattaactactggtaactgtaattgggcgct | |
| aagtctagtaga | |
| 8.45 NCZ 333 | |
| gcaagattgggagaccctttgacattggagcaaggc | (SEQ ID NO:12) |
| gctcaagaagttagagaaggtgacggtacaagggtc | |
| tcagaaattaactactggtaactgtaattgggcgct | |
| aagtctagtaga | |
| UI01866 638 | |
| cttatttcat-gataccaactttgtaaaagaaaagg | (SEQ ID NO:13) |
| actggcagctgagggat-gtcattccattgctggaa | |
| gat-gtaactcagacgctgtcaggacaagaaagaga | |
| ggcctttgaaag | |
| 8.45 NCZ 453 | |
| cttatttcattgataccaactttgtaaaagaaaagg | (SEQ ID NO:14) |
| actggcagctgagggattgtcattccattgctggaa | |
| gattgtaactcagacgctgtcaggacaagaaagaga | |
| ggcctttgaaag | |
| UIO1866 755 | |
| aacat-ggtgggcaatttctgctgtaaagat-gggc | (SEQ ID NO:15) |
| ctccagattaataat-gtagtagat-ggaaaggcat | |
| cattccagctcctaagagcgaaatat-gaaaagaag | |
| actgctaataaa | |
| 8.45 NCZ 573 | |
| aacattggtgggcaatttctgctgtaaagattgggc | (SEQ ID NO:16) |
| ctccagattaataattgtagtagattggaaaggcat | |
| cattccagctcctaagagcgaaatattgaaaagaag | |
| actgctaataaa | |
| UI01866 870 | |
| aagcagtctgagccctctgaagaatatc | (SEQ ID NO:17) |
| 8.45 NCZ 693 | |
| aagcagtctgagccctctgaagaatatc | (SEQ ID NO:18) |

Codon Optimisation
Codon Optimisation of the SQ Version of B Domain Deleted Factor VIII

HepG2 cells were transduced with EIAV vectors expressing the wild type (WT) or the codon optimised (CO) ‘SQ’ version of the Factor VIII gene at two different MOIs (1× and 10×). Following passage of the transduced cells, fresh media was added and the cells incubated for 24 h. Media was removed and tested for Factor VIII activity using the COATEST (Chromogenix). In this assay the supernatant from cells transduced with the highest MOI of the vector containing the synthetic Factor VIII gene resulted in very high levels of activity (beyond the linear range of the assay). Comparing the WT×10 and CO×1 results there is a 50-fold increase in Factor VIII activity in cell supernatants as a result of codon-optimisation assuming there are ten-fold more vector copies in the WT-transduced cells.

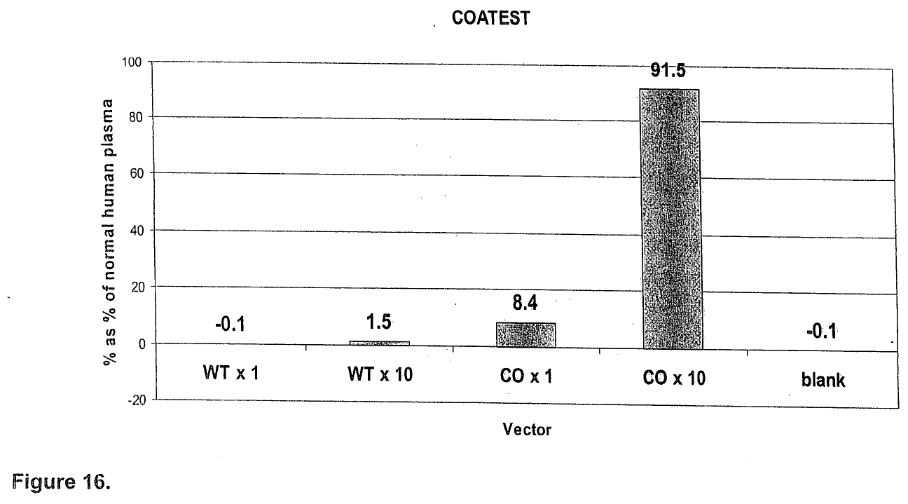

To test this, a real time PCR assay for EIAV Ψ signal was carried out on the transduced cells following passage. The assay detected approximately 2.5-fold more vector copies in the cells transduced with the CO vector compared to the WT vector. Codon-optimisation has therefore resulted in a 20-fold increase in Factor VIII activity (per vector copy). The results are shown in FIG. 16.

The experiment outlined in FIG. 16 was repeated and supernatants were split into two and appropriately diluted to assay for protein quantity (Affinity Biologicals FVIII ELISA) and activity (COATEST).

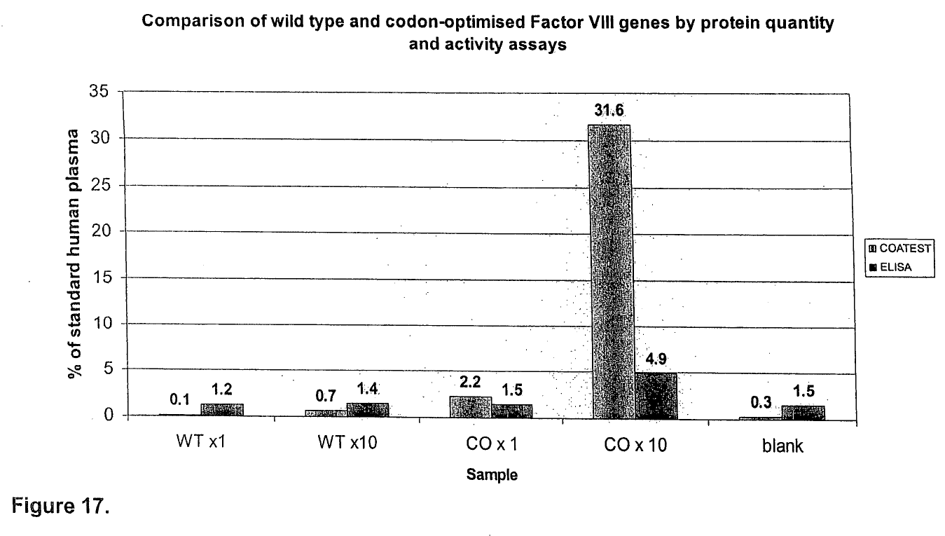

Although the Factor VIII activities are lower overall, again the codon-optimised samples had much greater levels of Factor VIII as measured by both assays. Only supernatant from the HepG2 cells transduced at the highest MOI gave a level of Factor VIII above background as measured by ELISA. This is likely due to the polyclonal primary antibody having being raised to full length Factor VIII protein and recognising epitopes on the full length protein which are missing on the B-domain deleted version. The results are shown in FIG. 17.

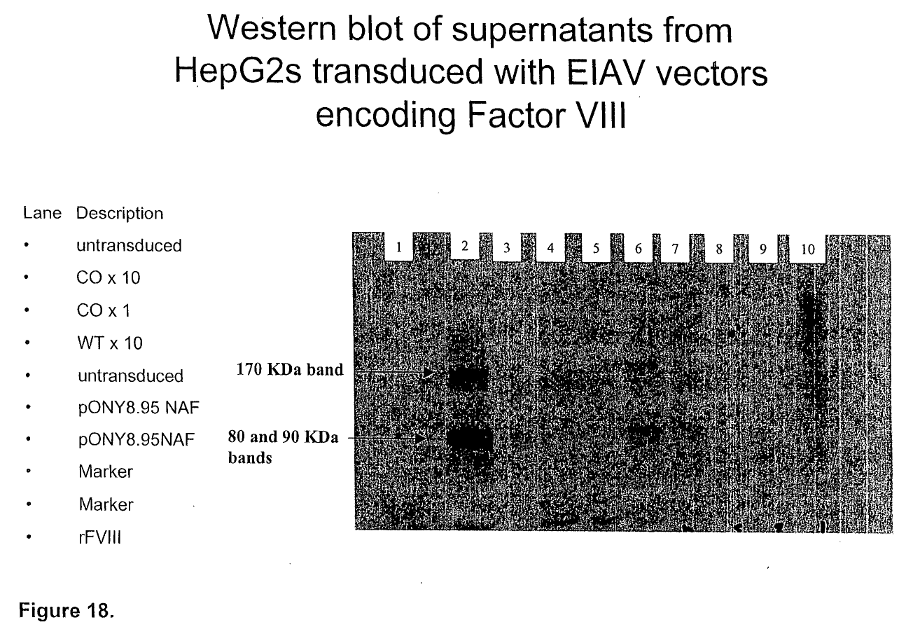

FIG. 18 shows a Western blot showing specific bands are present in the supernatant of cells transduced with the codon-optimised (CO) vector corresponding to the 170, 90 and 80 kDa Factor VIII polypeptides.

These bands are not present in either the untransduced supernatant, or supernatant from cells transduced with vector encoding the wild type Factor VIII gene.

Codon Optimisation of the Full Length Factor VIII Gene

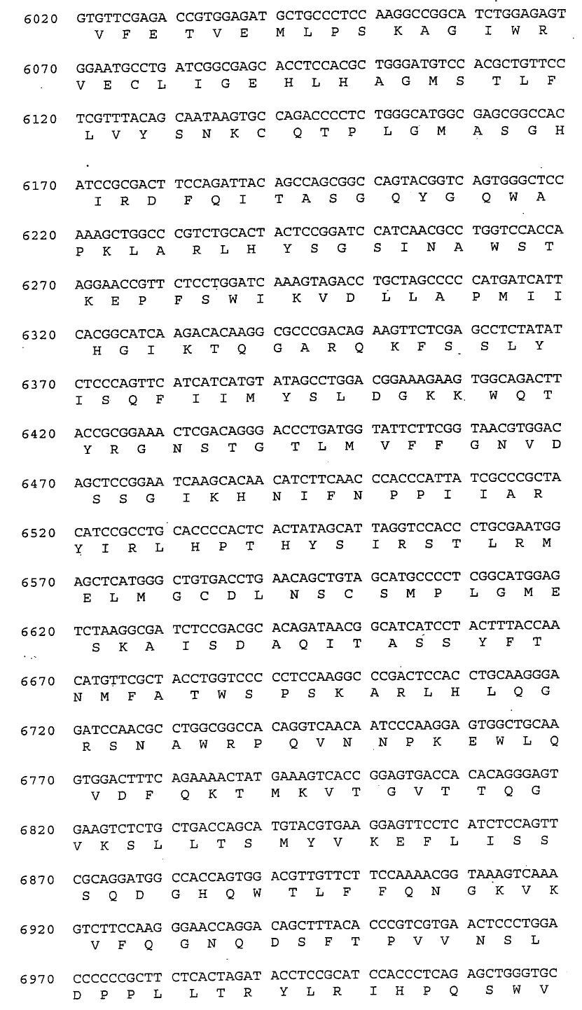

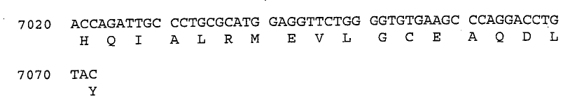

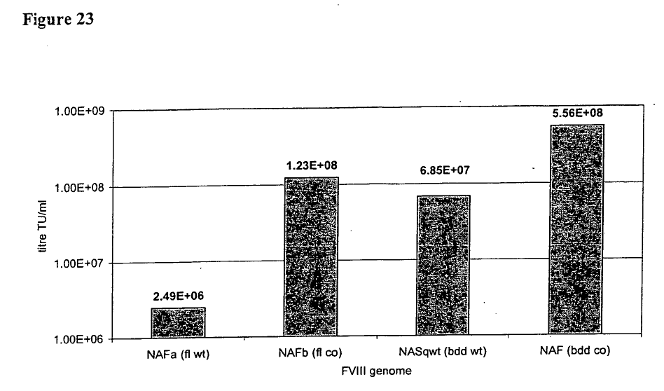

Viral vector was made by transient transfection of HEK293T cells and concentrated 2000-fold. HEK293T cells were then transduced with the indicated vectors (pRV67-pseudotyped). Following passaging and DNA extraction, EIAV Ψ levels were measured by real-time PCR and results expressed in the above graph as transducing units/ml (TU/ml). The results are shown in FIG. 23.

NAFa represents the full-length (fl), wild-type (wt) Factor VIII sequence; NAFb represents the full-length, codon-optimised (co) Factor VIII sequence; NASqwt represents the B-domain deleted (bdd), wild-type Factor VIII sequence; NAF represents the B-domain deleted, codon-optimised Factor VIII sequence. All genomes are in the pONY8.95 backbone.

Comparison of titres obtained from the full length sequences indicates that the codon-optimised version (NAFβ) produces titres 50 times greater than the wild-type version (NAFa). In addition, comparison of titres obtained from the B-domain deleted versions indicates that the codon-optimised version (NAF) produces titres 8 times greater than the wild-type version (NASqwt). Overall the B-domain deleted, codon-optimised version of the Factor VIII genome produces the highest titres.

Affect of Factor VIII Expression on Envelope

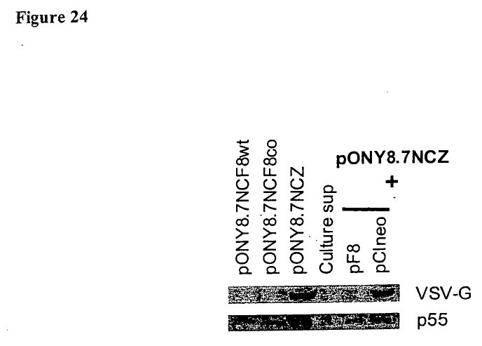

Expression of Factor VIII in producer cells clearly has a detrimental effect on vector titre. The reason for this discrepancy has previously been unclear. However, we have now shown that expression of Factor VIII in 293T producer cells results in a significant reduction of VSV-G envelope on the viral particles (see FIG. 24).

Factor VIII Inhibition of Viral Vector Production When Pseudotyped With Different Envelope Proteins

pONY8.95NCZ (LacZ genome) was prepared by transfection using optimised ratios of plasmid components including the various envelopes. To the transfection mix 2 μg of either pSQ (Factor VIII expressing plasmid) or pCIneo (control plasmid) was added. D17 titres (colony forming units (cfu)) are shown.

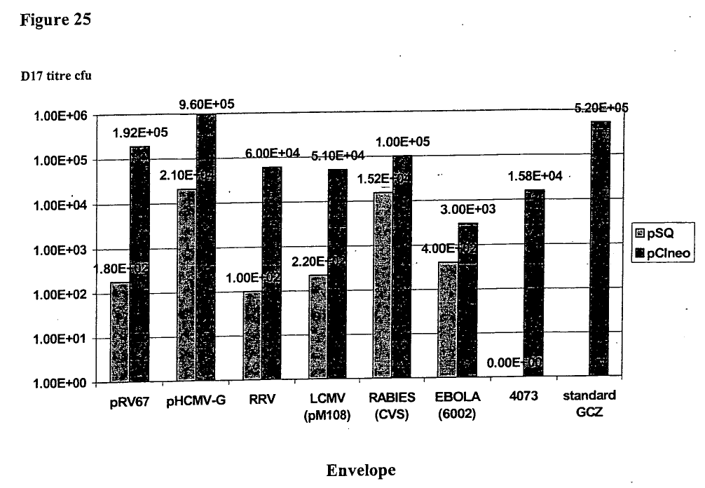

Several experiments have shown that Factor VIII expression has an inhibitory affect on viral vector production when pseudotyped with VSV-G (pRV67). To address whether the inhibition is specific to VSV-G the above experiment was performed using seven different envelopes (see FIG. 25). The results show that inhibition is not specific to VSV-G and that all titres are affected by Factor VIII expression to varying degrees. pHCMV-G appears to be less affected by Factor VIII expression than pRV67. This may be due to a single amino acid change on the second glycosylation site or could be due to a difference in expression levels.

The invention is further described by the following numbered paragraphs:

1. A lentiviral vector capable of delivering a nucleotide of interest (NOI) to a desired target site and wherein the NOI encodes for Factor VIII, or a derivative thereof, and the Factor VIII is expressed following delivery of the NOI to the desired target site.

2. A lentiviral vector comprising an NOI encoding for Factor VIII or a derivative thereof wherein the NOI is operably linked to a tissue specific promoter.

3. A lentiviral vector according to paragraph 2 wherein the tissue-specific promoter is a hepatic or endothelial tissue-specific promoter.

4. A lentiviral vector according to any preceding paragraph wherein the NOI is codon-optimised for expression in mammalian cells.

5. A lentiviral vector according to any preceding paragraph wherein the NOI is a B-domain deleted Factor VIII gene.

6. A retroviral vector comprising an NOI encoding for Factor VIII or a derivative thereof wherein the NOI is codon-optimised for expression in mammalian cells.

7. A vector according to paragraph 6 wherein the NOI is operably linked to a tissue specific promoter.

8. A vector according to paragraph 7 wherein the tissue-specific promoter is a hepatic or endothelial tissue-specific promoter.

9. A retroviral vector capable of delivering a first nucleotide of interest (NOI) and derivable from a retroviral pro-vector, wherein the retroviral pro-vector comprises a first NOI operably linked to an internal promoter and a second NOI between the first NOI and the internal promoter such that the second NOI is capable of being spliced out, and further wherein the promoter, first NOI and second NOI are in reverse complement orientation and optionally wherein the second NOI is optionally out of frame with respect to the first NOI.

10. A vector according to paragraph 9 wherein the second NOI is an intron optionally comprising at least part of an open reading frame (ORF).

11. A vector according to paragraph 9 or 10 wherein the retroviral pro-vector comprises a first nucleotide sequence (NS) capable of yielding a functional splice donor site and a second NS capable of yielding a functional splice acceptor site flanking the second NOI, and wherein the functional splice donor site is upstream of the functional splice acceptor site.

12. A vector according to any one of paragraphs 9 to 11 wherein the first NOI, or expression product thereof, is or comprises a therapeutic agent or a diagnostic agent.

13. A vector according to paragraph 12 wherein the expression product of the first NOI is Factor VIII.

14. A vector according to paragraph 13 wherein the Factor VIII is codon-optimised for expression in mammalian cells.

15. A vector according to any one of paragraphs 9 to 14 wherein the first NOI is operably linked to a tissue-specific promoter.

16. A vector according to paragraph 15 wherein the tissue-specific promoter is a hepatic or endothelial tissue-specific promoter.

17. A vector according to any one of paragraphs 9 to 16 wherein the second NOI, or expression product thereof, is or comprises any one or more of an agent conferring selectability (e.g. a marker element), a viral essential element, or part thereof, or combinations thereof.

18. A vector according to any one of paragraphs 9 to 17 wherein the second NOI includes a polyadenylation signal.

19. A vector according to any preceding paragraph wherein the vector or pro-vector is derivable from a lentivirus.

20. A vector according to any preceding paragraph wherein the lentivirus is HIV-1 or EIAV.

21. A vector according to any preceding paragraph wherein the vector is pseudotyped.

22. A vector according to any preceding paragraph wherein the vector is pseudotyped with VSV-G, a Ross River viral envelope or GP64.

23. A vector according any preceding paragraph to further comprising a Woodchuck hepatitis posttranscriptional element (WPRE).

24. A retroviral vector wherein the major splice donor is absent or disrupted.

25. A retroviral vector according to paragraph 24 wherein the retroviral vector is a lentiviral vector.

26. A vector according to any one of paragraphs 21 to 23 wherein the major splice donor is absent or disrupted.

27. A retroviral vector wherein the initial codon of the Tat exon is disrupted.

28. A retroviral vector according to paragraph 27 wherein the retroviral vector is a lentiviral vector.

29. A retroviral vector according any one of paragraphs 21 to 26 wherein the initial codon of the Tat exon is disrupted.

30. A lentiviral vector pseudotyped with a Ross River viral envelope wherein the lentiviral vector is derivable from HIV-1 or EIAV.

31. A lentiviral vector derivable from a lentiviral pro-vector, wherein the Tat exon of lentiviral pro-vector is deleted or disrupted such that the at least part of the Tat protein is not expressed in a target cell.

32. A retroviral vector derivable from a retroviral pro-vector, wherein the major splice donor is absent or disrupted.

33. A retroviral vector as defined in any one of the preceding paragraphs wherein the retroviral vector is an integrated provirus.

34. A retroviral particle obtainable from a retroviral vector according to any one of the preceding paragraphs.

35. A cell transfected or transduced with a retroviral vector according to any one of paragraphs 1-33 or a retroviral particle according to paragraph 34.

36. A retroviral vector according to any one of paragraphs 1-33 or a viral particle according to paragraph 34 or a cell according to paragraph 35 for use in medicine.

37. Use of a retroviral vector according to any one of paragraphs 1-33 or a viral particle according to paragraph 34 or a cell according to paragraph 35 for the preparation of a medicament to deliver one or more NOIs to a target site in need of same.

38. A method comprising transfecting or transducing a cell with retroviral vector according to any one of paragraphs 1-33 or a viral particle according to paragraph 34 or by use of a cell according to paragraph 35.

39. A method for producing Factor VIII in vitro comprising generating a cell as described in paragraph 35, passaging said cell in media, removing said media and isolating Factor VIII.

40. A method for producing Factor VIII in vitro comprising generating a cell comprising a codon optimised nucleic acid encoding Factor VIII in accordance with the invention, passaging said cell in media, removing said media and isolating Factor VIII.

Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A lentiviral vector comprising a nucleotide of interest (NOI) encoding Factor VIII, wherein said NOI is operably linked to a tissue specific promoter, and wherein the NOI is codon-optimised for expression in mammalian cells.

2. The lentiviral vector of claim 1, wherein the tissue-specific promoter is a hepatic or endothelial tissue-specific promoter.

3. The lentiviral vector of claim 1, wherein the Factor VIII is B-domain deleted Factor VIII.

4. A retroviral pro-vector comprising a first NOI operably linked to an internal promoter and a second NOI, wherein the second NOI is between the first NOI and the internal promoter, wherein the internal promoter, first NOI and second NOI are in reverse complement orientation, and wherein prior to packaging of the retroviral pro-vector the second NOI is spliced.

5. The retroviral pro-vector of claim 4, wherein the second NOI is out of frame with respect to the first NOI.

6. The retroviral pro-vector of claim 4, wherein the second NOI is an intron.

7. The retroviral pro-vector of claim 6, wherein the intron comprises at least part of an open reading frame (ORF).

8. The retroviral pro-vector of claim 4, wherein the retroviral pro-vector comprises a first nucleotide sequence (NS) comprising a functional splice donor site and a second NS comprising a functional splice acceptor site, wherein the first NS and the second NS flank the second NOI and wherein the functional splice donor site is upstream of the functional splice acceptor site.

9. The retroviral pro-vector of claim 4, wherein the first NOI is a therapeutic NOI.

10. The retroviral pro-vector of claim 4, wherein the first NOI encodes Factor VIII.

11. The retroviral pro-vector of claim 10, wherein the first NOI is operably linked to a tissue-specific promoter.

12. The retroviral pro-vector of claim 11, wherein the tissue-specific promoter is a hepatic or endothelial tissue-specific promoter.

13. The retroviral pro-vector of claim 4, wherein the first NOI is codon optimised for expression in mammalian cells.

14. The retroviral pro-vector of claim 4, wherein the second NOI encodes a selectable marker or a viral essential element.

15. The retroviral pro-vector of claim 4, wherein the second NOI includes a polyadenylation signal.

16. The retroviral pro-vector of claim 4, wherein the retroviral pro-vector is a lentiviral pro-vector.

17. The retroviral pro-vector of claim 4, wherein the lentiviral pro-vector is an HIV-1-based lentiviral pro-vector or an EIAV-based lentiviral pro-vector.

18. The retroviral pro-vector of claim 4, wherein the retroviral pro-vector is capable of being pseudotyped with an env protein.

19. The retroviral pro-vector of claim 8, wherein the env protein is VSV G, Ross River, or gp64.

20. The retroviral pro-vector of claim 4, wherein the retroviral pro-vector comprises a Woodchuck hepatitis posttranscriptional element (WPRE).

21. The retroviral pro-vector of claim 4, wherein the retroviral pro-vector comprises a non-functional major splice donor.

22. The retroviral pro-vector of claim 21, wherein the non-functional major splice donor is absent or disrupted.

23. A lentiviral pro-vector comprising a non-functional Tat exon.

24. The lentiviral pro-vector of claim 23, wherein the non-functional Tat exon is deleted or disrupted.

25. The lentiviral pro-vector of claim 24, wherein the initial codon of the Tat exon is disrupted.

26. A method for transfecting or transducing a cell comprising contacting the retroviral pro-vector of claim 23 with the cell, thereby transfecting or transducing the cell.

27. A method for transfecting or transducing a cell comprising contacting the retroviral pro-vector of claim 10 with the cell, thereby transfecting or transducing the cell and expressing Factor VIII in the cell.

28. The method of claim 27, further comprising passaging the cell in media, removing the media from the cell, and isolating Factor VIII from the cell.

29. The method of claim 27, wherein the Factor VIII is encoded by an NOI which is codon optimised for expression in mammalian cells.

30. The method of claim 29, further comprising passaging the cell in media, removing the media from the cell, and isolating Factor VIII from the cell.

31. A method for transfecting or transducing a cell comprising contacting the lentiviral pro-vector of claim 4 with the cell, thereby transfecting or transducing the cell.

32. A method for treating a haemophilia patient in need thereof, comprising administering a lentiviral vector to a target site in the patient, wherein the lentiviral vector comprises an NOI encoding Factor VIII, wherein the target site comprises liver or blood cells, and wherein Factor VIII is expressed in the target site thereby treating the patient.

33. The method of claim 32, wherein the Factor VIII is B-domain deleted Factor VIII.

34. The method of claim 32, wherein the NOI is operably linked to a tissue-specific promoter.

35. The method of claim 34, wherein the tissue-specific promoter is a hepatic or endothelial tissue-specific promoter.

36. The method of claim 32, wherein the NOI is codon optimised for expression in mammalian cells.


SEQUENCE LISTING (24 sequences)

SEQ ID NO: 1 - DNA, 38 bp, Artificial Sequence
  Description of Artificial Sequence Synthetic primer
tatgagcggccgcgtacccgccaccccctccaccttgg

SEQ ID NO: 2 - DNA, 35 bp, Artificial Sequence
  Description of Artificial Sequence Synthetic primer
atcatgcacgtgttcactgtcccaggtcagtggtg

SEQ ID NO: 3 - DNA, 10 bp, Artificial Sequence
  Description of Artificial Sequence Synthetic oligonucleotide
gttgaacctg

SEQ ID NO: 4 - DNA, 11 bp, Artificial Sequence
  Description of Artificial Sequence Synthetic oligonucleotide
gttgaacctcg

SEQ ID NO: 5 - DNA, 62 bp, Artificial Sequence
  Description of Artificial Sequence Synthetic oligonucleotide
gggacctgagaggggcgcagaccctacctgttgaacctcggctgatcgtaggatccccggga

SEQ ID NO: 6 - DNA, 60 bp, Artificial Sequence
  Description of Artificial Sequence Synthetic oligonucleotide
tgtaagttctcctctgctgtcccggggatcctacgatcagccgaggttcaacaggtaggg

SEQ ID NO: 7 - DNA, 39 bp, Artificial Sequence
  Description of Artificial Sequence Synthetic oligonucleotide
cagaacacaggaggacaggcaagattgggagaccctttg

SEQ ID NO: 8 - DNA, 39 bp, Artificial Sequence
  Description of Artificial Sequence Synthetic oligonucleotide
caaagggtctcccaatcttgcctgtcctcctgtgttctg

SEQ ID NO: 9 - DNA, 119 bp, Artificial Sequence
  Description of Artificial Sequence Synthetic nucleotide sequence
cctgagaggggcgcagaccctacctgttgaacctggctgatcgtaggatccccgggacagcagaggagaacttacagaagtcttctggaggtgttcctggccagaacacaggaggacag

SEQ ID NO: 10 - DNA, 120 bp, Artificial Sequence
  Description of Artificial Sequence Synthetic nucleotide sequence
cctgagaggggcgcagaccctacctgttgaacctcggctgatcgtaggatccccgggacagcagaggagaacttacagaagtcttctggaggtgttcctggccagaacacaggaggacag

SEQ ID NO: 11 - DNA, 118 bp, Artificial Sequence
  Description of Artificial Sequence Synthetic nucleotide sequence
gtaagatgggagaccctttgacatggagcaaggcgctcaagaagttagagaaggtgacggtacaagggtctcagaaattaactactggtaactgtaattgggcgctaagtctagtaga

SEQ ID NO: 12 - DNA, 120 bp, Artificial Sequence
  Description of Artificial Sequence Synthetic nucleotide sequence
gcaagattgggagaccctttgacattggagcaaggcgctcaagaagttagagaaggtgacggtacaagggtctcagaaattaactactggtaactgtaattgggcgctaagtctagtaga

SEQ ID NO: 13 - DNA, 117 bp, Artificial Sequence
  Description of Artificial Sequence Synthetic nucleotide sequence
cttatttcatgataccaactttgtaaaagaaaaggactggcagctgagggatgtcattccattgctggaagatgtaactcagacgctgtcaggacaagaaagagaggcctttgaaag

SEQ ID NO: 14 - DNA, 120 bp, Artificial Sequence
  Description of Artificial Sequence Synthetic nucleotide sequence
cttatttcattgataccaactttgtaaaagaaaaggactggcagctgagggattgtcattccattgctggaagattgtaactcagacgctgtcaggacaagaaagagaggcctttgaaag

SEQ ID NO: 15 - DNA, 115 bp, Artificial Sequence
  Description of Artificial Sequence Synthetic nucleotide sequence
aacatggtgggcaatttctgctgtaaagatgggcctccagattaataatgtagtagatggaaaggcatcattccagctcctaagagcgaaatatgaaaagaagactgctaataaa

SEQ ID NO: 16 - DNA, 120 bp, Artificial Sequence
  Description of Artificial Sequence Synthetic nucleotide sequence
aacattggtgggcaatttctgctgtaaagattgggcctccagattaataattgtagtagattggaaaggcatcattccagctcctaagagcgaaatattgaaaagaagactgctaataaa

SEQ ID NO: 17 - DNA, 28 bp, Artificial Sequence
  Description of Artificial Sequence Synthetic nucleotide sequence
aagcagtctgagccctctgaagaatatc

SEQ ID NO: 18 - DNA, 28 bp, Artificial Sequence
  Description of Artificial Sequence Synthetic nucleotide sequence
aagcagtctgagccctctgaagaatatc

SEQ ID NO: 19 - protein, 26 aa, Artificial Sequence
  Description of Artificial Sequence Synthetic protein sequence
RSFSQNSRHRSTRQKQFNATTIPEND

SEQ ID NO: 20 - protein, 65 aa, Artificial Sequence
  Description of Artificial Sequence Synthetic protein sequence
TERLCSQNPPVLKRHQREITRTTLQSDQEEIDYDDTISVEMKKEDFDIYDEDENQSPRSFQKKTR

SEQ ID NO: 21 - DNA, 4371 bp, Artificial Sequence
  Description of Artificial Sequence Synthetic nucleotide sequence
atgcagatcgaactgagcacttgcttcttcctgtgtctcctgcgcttttgcttctccgccacaaggagatactatctcggtgccgtggagctcagctgggactacatgcagagcgacttgggtgaactgcctgtggacgccaggtttccaccccgcgtgcccaagagtttcccgttcaacaccagtgtcgtgtacaagaaaaccctcttcgtggaattcaccgaccacctgttcaacatcgccaaaccgcgccctccctggatggggctgctcggcccgacgatccaggctgaggtctatgacacggtggtgattaccctcaagaacatggctagccacccggtgagcctgcacgccgtgggcgtgtcctattggaaagcgtccgagggtgcggagtacgatgaccagacttcacagcgggagaaggaagacgacaaagtgttccccgggggttcccacacctatgtctggcaggtcctgaaggagaatggtcctatggcctccgacccattgtgcctcacctactcttacctaagccatgtggatctcgtcaaggacctgaactcggggctgatcggcgccctgctcgtgtgccgggagggctcactggccaaggagaagacccaaactctgcacaagttcatcctgctgttcgcggtattcgacgaggggaagtcctggcactccgagaccaagaacagcctgatgcaggaccgcgacgcagcctcggcccgtgcgtggccaaagatgcacaccgtgaacggctacgttaacaggagcctacccggcctgatcggctgccaccgcaaatcggtctactggcatgtgatcggaatgggcacaacgcccgaggtccacagtatcttcctcgagggccacactttcctggtccggaatcaccgccaggccagcctggagatcagccccataacctttctgacggcgcagaccttactcatggatctcggccagttcctcctgttctgccacatttcgtcccaccagcacgatgggatggaagcatatgtgaaagtggactcctgccccgaggaaccccagcttaggatgaagaacaatgaggaggccgaggactacgacgatgaccttaccgattcagaaatggacgtagtacgctttgacgacgacaactctccatccttcatacagattcgctccgtcgccaagaagcaccctaagacttgggtgcactacatcgcggccgaggaggaggactgggattatgctcccctggtgctggcccccgacgaccgcagctacaagagccagtacctgaataacgggccccagcgcatcggccggaagtacaagaaagtgcggttcatggcttacacggacgagaccttcaagacccgggaggctatccagcatgagagcggcatcttggggcccctcctgtacggcgaagttggagacacactgctgatcatcttcaagaaccaggcgagcaggccctacaacatctacccccacggcattaccgatgtccggccgttgtacagccgacggctgcccaagggcgtgaagcacctgaaggactttccgatcctgccgggcgagatcttcaagtacaagtggactgtgaccgtggaggatgggccgaccaagagcgatccgcgctgcctgacccgttactactccagctttgtcaatatggagcgcgacctcgctagcggcttgattggccctctgctgatctgctacaaggagtccgtggaccagagggggaatcagatcatgagtgacaagaggaacgtgatcctgttctccgtgttcgacgaaaaccgcagctggtatctcaccgagaatatccagcgcttcctgcccaacccggccggtgtgcagctggaggaccccgagtttcaggccagcaacatcatgcattctatcaacggatatgtgtttgattccctgcagctctcagtgtgtctgcacgaggtcgcctactggtatatcctcagcattggggcacagaccgacttcctgagcgtgttcttctccgggtataccttcaagcacaagatggtgtacgaggataccctgaccctgttcccctttagcggcgaaaccgtgtttatgtctatggagaaccccgggctctggatccttggctgccataactccgacttccgcaaccgcggaatgaccgcgctcctgaaagtgtcgagttgtgacaagaacaccggcgactattacgaggacagttacgaggacatctctgcgtacctccttagcaagaataacgccatcgagccaagatccttcagccagaaccccccagtgctgaagaggcatcagcgggagatcacccgcacgaccctgcagtcggatcaggaggagattgattacgacgacacgatcagtgtggagatgaagaaggaggacttcgacatctacgacgaagatgaaaaccagtcccctcggtccttccaaaagaagacccggcactacttcatcgccgctgtggaacgcctgtgggactatggaatgtcttctagccctcacgttttgaggaaccgcgcccagtcgggcagcgtgccccagttcaagaaagtggtgttccaggagttcaccgacggctccttcacccagccactttaccggggcgagctcaatgaacatctgggcctgctgggaccctacatcagggctgaggtggaggacaacatcatggtgacattccggaatcaggccagcagaccatacagtttctacagttcactcatctcctacgaggaggaccagcgccagggggctgaaccccgtaagaacttcgtgaagccaaacgaaacaaagacctacttctggaaggtccagcaccacatggcacctaccaaggacgagttcgattgcaaggcctgggcctacttctccgacgtggacctggagaaagatgtgcacagcggcctgattggccctctgctggtgtgtcacacgaacacactcaaccctgcacacgggcggcaggtcactgtgcaggaattcgccctgttctttaccatctttgatgagacgaagtcctggtatttcaccgaaaacatggagaggaactgccgcgcaccctgcaacatccagatggaagatccgacattcaaggagaactaccggttccatgccatcaatggctacatcatggacaccctgcctggcctcgtgatggcccaagaccagcgtatccgctggtatctgctgtcgatgggctccaacgagaacatccatagtatccacttcagcgggcatgtcttcacggtgaggaaaaaggaggagtacaagatggcactgtacaacctctatcccggcgtgttcgagaccgtggagatgctgccctccaaggccggcatctggagagtggaatgcctgatcggcgagcacctccacgctgggatgtccacgctgttcctcgtttacagcaataagtgccagacccctctgggcatggcgagcggccacatccgcgacttccagattacagccagcggccagtacggtcagtgggctccaaagctggcccgtctgcactactccggatccatcaacgcctggtccaccaaggaaccgttctcctggatcaaagtagacctgctagcccccatgatcattcacggcatcaagacacaaggcgcccgacagaagttctcgagcctctatatctcccagttcatcatcatgtatagcctggacggaaagaagtggcagacttaccgcggaaactcgacagggaccctgatggtattcttcggtaacgtggacagctccggaatcaagcacaacatcttcaacccacccattatcgcccgctacatccgcctgcaccccactcactatagcattaggtccaccctgcgaatggagctcatgggctgtgacctgaacagctgtagcatgcccctcggcatggagtctaaggcgatctccgacgcacagataacggcatcatcctactttaccaacatgttcgctacctggtccccctccaaggcccgactccacctgcaagggagatccaacgcctggcggccacaggtcaacaatcccaaggagtggctgcaagtggactttcagaaaactatgaaagtcaccggagtgaccacacagggagtgaagtctctgctgaccagcatgtacgtgaaggagttcctcatctccagttcgcaggatggccaccagtggacgttgttcttccaaaacggtaaagtcaaagtcttccaagggaaccaggacagctttacacccgtcgtgaactccctggaccccccgcttctcactagatacctccgcatccaccctcagagctgggtgcaccagattgccctgcgcatggaggttctggggtgtgaagcccaggacctgtac

SEQ ID NO: 22 - DNA, 13552 bp, Artificial Sequence
  Description of Artificial Sequence Synthetic nucleotide sequence
agcttcacgtgccgccaccatgcagatcgaactgagcacttgcttcttcctgtgtctcctgcgcttttgcttctccgccacaaggagatactatctcggtgccgtggagctcagctgggactacatgcagagcgacttgggtgaactgcctgtggacgccaggtttccaccccgcgtgcccaagagtttcccgttcaacaccagtgtcgtgtacaagaaaaccctcttcgtggaattcaccgaccacctgttcaacatcgccaaaccgcgccctccctggatggggctgctcggcccgacgatccaggctgaggtctatgacacggtggtgattaccctcaagaacatggctagccacccggtgagcctgcacgccgtgggcgtgtcctattggaaagcgtccgagggtgcggagtacgatgaccagacttcacagcgggagaaggaagacgacaaagtgttccccgggggttcccacacctatgtctggcaggtcctgaaggagaatggtcctatggcctccgacccattgtgcctcacctactcttacctaagccatgtggatctcgtcaaggacctgaactcggggctgatcggcgccctgctcgtgtgccgggagggctcactggccaaggagaagacccaaactctgcacaagttcatcctgctgttcgcggtattcgacgaggggaagtcctggcactccgagaccaagaacagcctgatgcaggaccgcgacgcagcctcggcccgtgcgtggccaaagatgcacaccgtgaacggctacgttaacaggagcctacccggcctgatcggctgccaccgcaaatcggtctactggcatgtgatcggaatgggcacaacgcccgaggtccacagtatcttcctcgagggccacactttcctggtccggaatcaccgccaggccagcctggagatcagccccataacctttctgacggcgcagaccttactcatggatctcggccagttcctcctgttctgccacatttcgtcccaccagcacgatgggatggaagcatatgtgaaagtggactcctgccccgaggaaccccagcttaggatgaagaacaatgaggaggccgaggactacgacgatgaccttaccgattcagaaatggacgtagtacgctttgacgacgacaactctccatccttcatacagattcgctccgtcgccaagaagcaccctaagacttgggtgcactacatcgcggccgaggaggaggactgggattatgctcccctggtgctggcccccgacgaccgcagctacaagagccagtacctgaataacgggccccagcgcatcggccggaagtacaagaaagtgcggttcatggcttacacggacgagaccttcaagacccgggaggctatccagcatgagagcggcatcttggggcccctcctgtacggcgaagttggagacacactgctgatcatcttcaagaaccaggcgagcaggccctacaacatctacccccacggcattaccgatgtccggccgttgtacagccgacggctgcccaagggcgtgaagcacctgaaggactttccgatcctgccgggcgagatcttcaagtacaagtggactgtgaccgtggaggatgggccgaccaagagcgatccgcgctgcctgacccgttactactccagctttgtcaatatggagcgcgacctcgctagcggcttgattggccctctgctgatctgctacaaggagtccgtggaccagagggggaatcagatcatgagtgacaagaggaacgtgatcctgttctccgtgttcgacgaaaaccgcagctggtatctcaccgagaatatccagcgcttcctgcccaacccggccggtgtgcagctggaggaccccgagtttcaggccagcaacatcatgcattctatcaacggatatgtgtttgattccctgcagctctcagtgtgtctgcacgaggtcgcctactggtatatcctcagcattggggcacagaccgacttcctgagcgtgttcttctccgggtataccttcaagcacaagatggtgtacgaggataccctgaccctgttcccctttagcggcgaaaccgtgtttatgtctatggagaaccccgggctctggatccttggctgccataactccgacttccgcaaccgcggaatgaccgcgctcctgaaagtgtcgagttgtgacaagaacaccggcgactattacgaggacagttacgaggacatctctgcgtacctccttagcaagaataacgccatcgagccaagatccttcagccagaacagccggcaccccagcacccggcagaagcagttcaacgccaccaccatccccgagaacgacatcgagaaaaccgacccctggttcgcccaccggacccccatgcccaagatccagaacgtgagcagcagcgacctgctgatgctgctgcggcagagccccaccccccacggcctgagcctgagcgacctgcaggaggccaagtacgagaccttcagcgacgaccccagccctggcgccatcgacagcaacaacagcctgtccgagatgacccacttccggccccagctgcaccacagcggcgacatggtgttcacccccgagagcggcctgcagctgcggctgaacgagaagctgggcaccaccgccgccaccgagctgaagaagctggacttcaaagtgagcagcaccagcaacaacctgatcagcaccatccccagcgacaacctggccgccggcaccgacaacaccagcagcctgggccctcccagcatgcccgtgcactacgacagccagctggacaccaccctgttcggcaagaagagcagccccctgacagagagcggcggacccctgagcctgtctgaggagaacaacgacagcaagctgctggagtccggcctgatgaacagccaggagtccagctggggcaagaacgtgtctagcaccgagagcggacggctgttcaagggcaagcgggcccacggccctgccctgctgaccaaggacaacgccctgttcaaagtgtccatcagcctgctgaaaaccaacaagacctccaacaacagcgccaccaaccgcaagacccacatcgacggcccaagcctgctgatcgagaacagccccagcgtgtggcagaacatcctggagagcgacaccgagttcaagaaagtgacccccctgatccacgaccggatgctgatggataagaacgccaccgccctgagactgaaccacatgagcaacaagaccacctccagcaagaacatggagatggtgcagcagaagaaggagggccccatcccccccgacgcccagaaccccgacatgagcttcttcaagatgctgttcctgcccgagagcgcccggtggatccagcggacccacggcaagaacagcctgaacagcggccagggccccagccccaagcagctggtgagcctgggacccgagaagagcgtggagggccagaacttcctgagcgagaagaacaaagtggtggtgggcaagggcgagttcaccaaggatgtgggcctgaaggagatggtgttccccagcagccggaacctgttcctgaccaacctggacaacctgcacgagaacaacacccacaaccaggagaagaagatccaggaggagatcgagaagaaggaaaccctgatccaggagaacgtggtgctgccccagatccacaccgtgaccggcaccaagaacttcatgaagaatctgttcctgctgagcaccagacagaacgtggagggcagctacgacggcgcctacgcccccgtgctgcaggacttccggagcctgaacgacagcaccaaccggaccaagaagcacaccgcccacttcagcaagaagggcgaggaggagaacctggagggcctgggcaaccagaccaagcagatcgtggagaagtacgcctgcaccacccggatcagccccaacaccagccagcagaacttcgtgacccagcggagcaagagagccctgaagcagtttcggctgcccctggaggagacagagctggagaagcggatcatcgtggacgacaccagcacacagtggtccaagaacatgaagcacctgacccctagcaccctgacccagatcgactacaacgagaaggagaagggcgccatcacccagagccccctgagcgactgcctgacccggagccacagcatcccccaggccaaccggagccccctgcctatcgccaaagtgtctagcttccccagcatcaggcccatctacctgaccagagtgctgttccaggacaacagctcccacctgcctgccgccagctaccggaagaaggacagcggcgtgcaggagagcagccacttcctgcagggcgccaagaagaacaacctgagcctggccatcctgaccctggagatgaccggcgaccagcgggaagtgggcagcctgggaaccagcgccacaaacagcgtgacctacaagaaagtggagaacaccgtgctgcccaagcccgacctgcccaagaccagcggaaaagtggagctgctgcccaaagtgcacatctaccagaaggacctgttccccaccgagaccagcaacggcagccctggccacctggacctggtggagggctccctgctgcagggcaccgagggcgccattaagtggaacgaggccaacagacccggcaaagtgcccttcctgagagtggccaccgagagcagcgccaagaccccctccaaactgctggaccccctggcctgggacaatcactacggcacccagatccccaaggaggagtggaagagccaggagaagtcccccgaaaagaccgccttcaagaagaaggataccatcctgtccctgaacgcctgcgagagcaaccacgccatcgccgccatcaacgagggacagaacaagcccgagatagaggtgacctgggcgaagcagggcagaaccgagcgcctgtgcagccagaaccccccagtgctgaagaggcatcagcgggagatcacccgcacgaccctgcagtcggatcaggaggagattgattacgacgacacgatcagtgtggagatgaagaaggaggacttcgacatctacgacgaagatgaaaaccagtcccctcggtccttccaaaagaagacccggcactacttcatcgccgctgtggaacgcctgtgggactatggaatgtcttctagccctcacgttttgaggaaccgcgcccagtcgggcagcgtgccccagttcaagaaagtggtgttccaggagttcaccgacggctccttcacccagccactttaccggggcgagctcaatgaacatctgggcctgctgggaccctacatcagggctgaggtggaggacaacatcatggtgacattccggaatcaggccagcagaccatacagtttctacagttcactcatctcctacgaggaggaccagcgccagggggctgaaccccgtaagaacttcgtgaagccaaacgaaacaaagacctacttctggaaggtccagcaccacatggcacctaccaaggacgagttcgattgcaaggcctgggcctacttctccgacgtggacctggagaaagatgtgcacagcggcctgattggccctctgctggtgtgtcacacgaacacactcaaccctgcacacgggcggcaggtcactgtgcaggaattcgccctgttctttaccatctttgatgagacgaagtcctggtatttcaccgaaaacatggagaggaactgccgcgcaccctgcaacatccagatggaagatccgacattcaaggagaactaccggttccatgccatcaatggctacatcatggacaccctgcctggcctcgtgatggcccaagaccagcgtatccgctggtatctgctgtcgatgggctccaacgagaacatccatagtatccacttcagcgggcatgtcttcacggtgaggaaaaaggaggagtacaagatggcactgtacaacctctatcccggcgtgttcgagaccgtggagatgctgccctccaaggccggcatctggagagtggaatgcctgatcggcgagcacctccacgctgggatgtccacgctgttcctcgtttacagcaataagtgccagacccctctgggcatggcgagcggccacatccgcgacttccagattacagccagcggccagtacggtcagtgggctccaaagctggcccgtctgcactactccggatccatcaacgcctggtccaccaaggaaccgttctcctggatcaaagtagacctgctagcccccatgatcattcacggcatcaagacacaaggcgcccgacagaagttctcgagcctctatatctcccagttcatcatcatgtatagcctggacggaaagaagtggcagacttaccgcggaaactcgacagggaccctgatggtattcttcggtaacgtggacagctccggaatcaagcacaacatcttcaacccacccattatcgcccgctacatccgcctgcaccccactcactatagcattaggtccaccctgcgaatggagctcatgggctgtgacctgaacagctgtagcatgcccctcggcatggagtctaaggcgatctccgacgcacagataacggcatcatcctactttaccaacatgttcgctacctggtccccctccaaggcccgactccacctgcaagggagatccaacgcctggcggccacaggtcaacaatcccaaggagtggctgcaagtggactttcagaaaactatgaaagtcaccggagtgaccacacagggagtgaagtctctgctgaccagcatgtacgtgaaggagttcctcatctccagttcgcaggatggccaccagtggacgttgttcttccaaaacggtaaagtcaaagtcttccaagggaaccaggacagctttacacccgtcgtgaactccctggaccccccgcttctcactagatacctccgcatccaccctcagagctgggtgcaccagattgccctgcgcatggaggttctggggtgtgaagcccaggacctgtactaatgatatcaagcttaaaaggtaccaaatagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgaattaattcgcggccctagcttatcgataccgtcgaattggaagagctttaaatcctggcacatctcatgtatcaatgcctcagtatgtttagaaaaacaaggggggaactgtggggtttttatgaggggttttatacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccgggagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggcagatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctcggctgatcgtaggatccccgggacagcagaggagaacttacagaagtcttctggaggtgttcctggccagaacacaggaggacaggcaagattgggagaccctttgacattggagcaaggcgctcaagaagttagagaaggtgacggtacaagggtctcagaaattaactactggtaactgtaattgggcgctaagtctagtagacttatttcattgataccaactttgtaaaagaaaaggactggcagctgagggattgtcattccattgctggaagattgtaactcagacgctgtcaggacaagaaagagaggcctttgaaagaacattggtgggcaatttctgctgtaaagattgggcctccagattaataattgtagtagattggaaaggcatcattccagctcctaagagcgaaatattgaaaagaagactgctaataaaaagcagtctgagccctctgaagaatatctctagaactagtggatcccccgggccaaaacctagcgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcggccgcgtacccgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaacacgcctggagacgccatccacgctgttttgacctccatagaagacaccgggaccgatccagcctccgcggcccca

SEQ ID NO: 23 - DNA, 7053 bp, Artificial Sequence
  CDS (1)..(7053)
  Description of Artificial Sequence Synthetic nucleotide sequence
atgcagatcgaactgagcacttgcttcttcctgtgtctcctgcgcttttgcttctccgccacaaggagatactatctcggtgccgtggagctcagctgggactacatgcagagcgacttgggtgaactgcctgtggacgccaggtttccaccccgcgtgcccaagagtttcccgttcaacaccagtgtcgtgtacaagaaaaccctcttcgtggaattcaccgaccacctgttcaacatcgccaaaccgcgccctccctggatggggctgctcggcccgacgatccaggctgaggtctatgacacggtggtgattaccctcaagaacatggctagccacccggtgagcctgcacgccgtgggcgtgtcctattggaaagcgtccgagggtgcggagtacgatgaccagacttcacagcgggagaaggaagacgacaaagtgttccccgggggttcccacacctatgtctggcaggtcctgaaggagaatggtcctatggcctccgacccattgtgcctcacctactcttacctaagccatgtggatctcgtcaaggacctgaactcggggctgatcggcgccctgctcgtgtgccgggagggctcactggccaaggagaagacccaaactctgcacaagttcatcctgctgttcgcggtattcgacgaggggaagtcctggcactccgagaccaagaacagcctgatgcaggaccgcgacgcagcctcggcccgtgcgtggccaaagatgcacaccgtgaacggctacgttaacaggagcctacccggcctgatcggctgccaccgcaaatcggtctactggcatgtgatcggaatgggcacaacgcccgaggtccacagtatcttcctcgagggccacactttcctggtccggaatcaccgccaggccagcctggagatcagccccataacctttctgacggcgcagaccttactcatggatctcggccagttcctcctgttctgccacatttcgtcccaccagcacgatgggatggaagcatatgtgaaagtggactcctgccccgaggaaccccagcttaggatgaagaacaatgaggaggccgaggactacgacgatgaccttaccgattcagaaatggacgtagtacgctttgacgacgacaactctccatccttcatacagattcgctccgtcgccaagaagcaccctaagacttgggtgcactacatcgcggccgaggaggaggactgggattatgctcccctggtgctggcccccgacgaccgcagctacaagagccagtacctgaataacgggccccagcgcatcggccggaagtacaagaaagtgcggttcatggcttacacggacgagaccttcaagacccgggaggctatccagcatgagagcggcatcttggggcccctcctgtacggcgaagttggagacacactgctgatcatcttcaagaaccaggcgagcaggccctacaacatctacccccacggcattaccgatgtccggccgttgtacagccgacggctgcccaagggcgtgaagcacctgaaggactttccgatcctgccgggcgagatcttcaagtacaagtggactgtgaccgtggaggatgggccgaccaagagcgatccgcgctgcctgacccgttactactccagctttgtcaatatggagcgcgacctcgctagcggcttgattggccctctgctgatctgctacaaggagtccgtggaccagagggggaatcagatcatgagtgacaagaggaacgtgatcctgttctccgtgttcgacgaaaaccgcagctggtatctcaccgagaatatccagcgcttcctgcccaacccggccggtgtgcagctggaggaccccgagtttcaggccagcaacatcatgcattctatcaacggatatgtgtttgattccctgcagctctcagtgtgtctgcacgaggtcgcctactggtatatcctcagcattggggcacagaccgacttcctgagcgtgttcttctccgggtataccttcaagcacaagatggtgtacgaggataccctgaccctgttcccctttagcggcgaaaccgtgtttatgtctatggagaaccccgggctctggatccttggctgccataactccgacttccgcaaccgcggaatgaccgcgctcctgaaagtgtcgagttgtgacaagaacaccggcgactattacgaggacagttacgaggacatctctgcgtacctccttagcaagaataacgccatcgagccaagatccttcagccagaacagccggcaccccagcacccggcagaagcagttcaacgccaccaccatccccgagaacgacatcgagaaaaccgacccctggttcgcccaccggacccccatgcccaagatccagaacgtgagcagcagcgacctgctgatgctgctgcggcagagccccaccccccacggcctgagcctgagcgacctgcaggaggccaagtacgagaccttcagcgacgaccccagccctggcgccatcgacagcaacaacagcctgtccgagatgacccacttccggccccagctgcaccacagcggcgacatggtgttcacccccgagagcggcctgcagctgcggctgaacgagaagctgggcaccaccgccgccaccgagctgaagaagctggacttcaaagtgagcagcaccagcaacaacctgatcagcaccatccccagcgacaacctggccgccggcaccgacaacaccagcagcctgggccctcccagcatgcccgtgcactacgacagccagctggacaccaccctgttcggcaagaagagcagccccctgacagagagcggcggacccctgagcctgtctgaggagaacaacgacagcaagctgctggagtccggcctgatgaacagccaggagtccagctggggcaagaacgtgtctagcaccgagagcggacggctgttcaagggcaagcgggcccacggccctgccctgctgaccaaggacaacgccctgttcaaagtgtccatcagcctgctgaaaaccaacaagacctccaacaacagcgccaccaaccgcaagacccacatcgacggcccaagcctgctgatcgagaacagccccagcgtgtggcagaacatcctggagagcgacaccgagttcaagaaagtgacccccctgatccacgaccggatgctgatggataagaacgccaccgccctgagactgaaccacatgagcaacaagaccacctccagcaagaacatggagatggtgcagcagaagaaggagggccccatcccccccgacgcccagaaccccgacatgagcttcttcaagatgctgttcctgcccgagagcgcccggtggatccagcggacccacggcaagaacagcctgaacagcggccagggccccagccccaagcagctggtgagcctgggacccgagaagagcgtggagggccagaacttcctgagcgagaagaacaaagtggtggtgggcaagggcgagttcaccaaggatgtgggcctgaaggagatggtgttccccagcagccggaacctgttcctgaccaacctggacaacctgcacgagaacaacacccacaaccaggagaagaagatccaggaggagatcgagaagaaggaaaccctgatccaggagaacgtggtgctgccccagatccacaccgtgaccggcaccaagaacttcatgaagaatctgttcctgctgagcaccagacagaacgtggagggcagctacgacggcgcctacgcccccgtgctgcaggacttccggagcctgaacgacagcaccaaccggaccaagaagcacaccgcccacttcagcaagaagggcgaggaggagaacctggagggcctgggcaaccagaccaagcagatcgtggagaagtacgcctgcaccacccggatcagccccaacaccagccagcagaacttcgtgacccagcggagcaagagagccctgaagcagtttcggctgcccctggaggagacagagctggagaagcggatcatcgtggacgacaccagcacacagtggtccaagaacatgaagcacctgacccctagcaccctgacccagatcgactacaacgagaaggagaagggcgccatcacccagagccccctgagcgactgcctgacccggagccacagcatcccccaggccaaccggagccccctgcctatcgccaaagtgtctagcttccccagcatcaggcccatctacctgaccagagtgctgttccaggacaacagctcccacctgcctgccgccagctaccggaagaaggacagcggcgtgcaggagagcagccacttcctgcagggcgccaagaagaacaacctgagcctggccatcctgaccctggagatgaccggcgaccagcgggaagtgggcagcctgggaaccagcgccacaaacagcgtgacctacaagaaagtggagaacaccgtgctgcccaagcccgacctgcccaagaccagcggaaaagtggagctgctgcccaaagtgcacatctaccagaaggacctgttccccaccgagaccagcaacggcagccctggccacctggacctggtggagggctccctgctgcagggcaccgagggcgccattaagtggaacgaggccaacagacccggcaaagtgcccttcctgagagtggccaccgagagcagcgccaagaccccctccaaactgctggaccccctggcctgggacaatcactacggcacccagatccccaaggaggagtggaagagccaggagaagtcccccgaaaagaccgccttcaagaagaaggataccatcctgtccctgaacgcctgcgagagcaaccacgccatcgccgccatcaacgagggacagaacaagcccgagatagaggtgacctgggcgaagcagggcagaaccgagcgcctgtgcagccagaaccccccagtgctgaagaggcatcagcgggagatcacccgcacgaccctgcagtcggatcaggaggagattgattacgacgacacgatcagtgtggagatgaagaaggaggacttcgacatctacgacgaagatgaaaaccagtcccctcggtccttccaaaagaagacccggcactacttcatcgccgctgtggaacgcctgtgggactatggaatgtcttctagccctcacgttttgaggaaccgcgcccagtcgggcagcgtgccccagttcaagaaagtggtgttccaggagttcaccgacggctccttcacccagccactttaccggggcgagctcaatgaacatctgggcctgctgggaccctacatcagggctgaggtggaggacaacatcatggtgacattccggaatcaggccagcagaccatacagtttctacagttcactcatctcctacgaggaggaccagcgccagggggctgaaccccgtaagaacttcgtgaagccaaacgaaacaaagacctacttctggaaggtccagcaccacatggcacctaccaaggacgagttcgattgcaaggcctgggcctacttctccgacgtggacctggagaaagatgtgcacagcggcctgattggccctctgctggtgtgtcacacgaacacactcaaccctgcacacgggcggcaggtcactgtgcaggaattcgccctgttctttaccatctttgatgagacgaagtcctggtatttcaccgaaaacatggagaggaactgccgcgcaccctgcaacatccagatggaagatccgacattcaaggagaactaccggttccatgccatcaatggctacatcatggacaccctgcctggcctcgtgatggcccaagaccagcgtatccgctggtatctgctgtcgatgggctccaacgagaacatccatagtatccacttcagcgggcatgtcttcacggtgaggaaaaaggaggagtacaagatggcactgtacaacctctatcccggcgtgttcgagaccgtggagatgctgccctccaaggccggcatctggagagtggaatgcctgatcggcgagcacctccacgctgggatgtccacgctgttcctcgtttacagcaataagtgccagacccctctgggcatggcgagcggccacatccgcgacttccagattacagccagcggccagtacggtcagtgggctccaaagctggcccgtctgcactactccggatccatcaacgcctggtccaccaaggaaccgttctcctggatcaaagtagacctgctagcccccatgatcattcacggcatcaagacacaaggcgcccgacagaagttctcgagcctctatatctcccagttcatcatcatgtatagcctggacggaaagaagtggcagacttaccgcggaaactcgacagggaccctgatggtattcttcggtaacgtggacagctccggaatcaagcacaacatcttcaacccacccattatcgcccgctacatccgcctgcaccccactcactatagcattaggtccaccctgcgaatggagctcatgggctgtgacctgaacagctgtagcatgcccctcggcatggagtctaaggcgatctccgacgcacagataacggcatcatcctactttaccaacatgttcgctacctggtccccctccaaggcccgactccacctgcaagggagatccaacgcctggcggccacaggtcaacaatcccaaggagtggctgcaagtggactttcagaaaactatgaaagtcaccggagtgaccacacagggagtgaagtctctgctgaccagcatgtacgtgaaggagttcctcatctccagttcgcaggatggccaccagtggacgttgttcttccaaaacggtaaagtcaaagtcttccaagggaaccaggacagctttacacccgtcgtgaactccctggaccccccgcttctcactagatacctccgcatccaccctcagagctgggtgcaccagattgccctgcgcatggaggttctggggtgtgaagcccaggacctgtac

SEQ ID NO: 24 - protein, 2351 aa, Artificial Sequence
  Description of Artificial Sequence Synthetic protein sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY